(19) United States
(12) Patent Application Publication
(10) Pub. No.: US 2023/0375528 A1
(43) Pub. Date: Nov. 23, 2023

(54) SCREENING METHOD FOR THE IDENTIFICATION OF NOVEL THERAPEUTIC COMPOUNDS

(71) Applicant: European Molecular Biology Laboratory, Heidelberg (DE)

(72) Inventors: Matthias W. HENTZE, Heidelberg (DE); Ina HUPPERTZ, Heidelberg (DE)

(73) Assignee: European Molecular Biology Laboratory, Heidelberg (DE)

(21) Appl. No.: 18/248,022

(22) PCT Filed: Oct. 6, 2021
(86) PCT No.: PCT/EP2021/077600

§ 371 Date: Apr. 5, 2023

(30) Foreign Application Priority Data
Oct. 6, 2020 (EP) 20200263.0

(51) Int. Cl. G01N 33/50; C12Q 1/527; C12N 9/88

(52) U.S. Cl. CPC G01N 33/5011; C12Q 1/527; C12N 9/88; C12Y 402/01011

(57) ABSTRACT

The present invention pertains to a method for identifying and/or characterizing a compound suitable for the prevention and/or treatment of a disease. The invention is based on the finding that the glycolytic enzyme Enolase 1 (ENO1) binds RNA, and its enzymatic activity is thereby regulated. The invention is further based on the finding that riboregulation of ENO1 affects cell differentiation, which plays a pivotal role in cancer. Accordingly, the invention provides a screening method for novel therapeutic compounds based on the binding of RNA to ENO1. Compounds screened according to the present invention can affect the binding of RNA to ENO1, which harbors the therapeutic potential for the treatment of diseases, in particular proliferative diseases, such as cancer. Methods of treatment using these compounds, as well as pharmaceutical compositions thereof, are also provided.

### FIELD OF THE INVENTION

The present invention pertains to a method for identifying and/or characterizing a compound suitable for the prevention and/or treatment of a disease. The invention is based on the finding that the glycolytic enzyme Enolase 1 (ENO1) binds RNA, and its enzymatic activity is thereby regulated. The invention is further based on the finding that riboregulation of ENO1 affects cell differentiation, which plays a pivotal role in cancer. Accordingly, the invention provides a screening method for novel therapeutic compounds based on the binding of RNA to ENO1. Compounds screened according to the present invention can affect the binding of RNA to ENO1, which harbors the therapeutic potential for the treatment of diseases, in particular proliferative diseases, such as cancer. Methods of treatment using these compounds, as well as pharmaceutical compositions thereof, are also provided.

### DESCRIPTION

Cancer is a major lethal disease for humans and is caused by physiologically-uncontrolled proliferation of tumor cells, which affects normal physiological conditions of the human body, and often ultimately leads to the death of patients. Although tremendous efforts on cancer studies and treatments have been made, cancer is still the major cause of death to humans. Current therapeutic options for most cancer diseases include radiation therapy to destroy cancer cells, chemotherapy to prevent tumorous cells from growing and dividing further, immunotherapy to boost the body's natural defense system to fight the cancer, and surgery to remove the tumorous tissue during an operation. However, all of these treatment strategies have severe side effects. Moreover, many cancers become resistant to treatment, or patients relapse following treatment. Furthermore, in patients treated with a chemotherapeutic agent, a continuous selection pressure exists to develop cancer cell clones which are resistant to the chemotherapeutic used.

Glycolysis represents a classic metabolic pathway that has received renewed interest in recent years due to its role in stem cell differentiation and cancer biology. Our fundamental understanding of glycolysis as a protein- and metabolite-controlled process has, however, remained widely unchanged over the last decades. One common feature in stem cell differentiation is the metabolic remodelling from aerobic glycolysis to respiration during the exit from pluripotency, with distinct paths taken by different germ layers.

Enolase 1 (ENO1), also known as alpha-enolase, is a glycolytic enzyme ubiquitously expressed in adult human tissues, including liver, brain, kidney, and spleen. ENO1 catalyses the reversible interconversion between 2-phosphoglycerate (2 PG) and phosphoenolpyruvate (PEP). ENO1 is a homodimer composed of 2 alpha subunits. Interestingly, ENO1 overexpression has been associated with multiple tumors, including glioma, neuroendocrine tumors, neuroblastoma, pancreatic cancer, prostate cancer, cholangiocarcinoma, thyroid carcinoma, lung cancer, hepatocellular carcinoma, and breast cancer. Further, ENO1 has been shown to be expressed on the tumor cell surface during pathological conditions, such as inflammation, autoimmunity, and malignancy. ENO1 also plays a role in other functions, including the role as cell surface receptor for plasminogen on pathogens, such as streptococci, and activated immune cells, leading to systemic infection or tissue invasion.

WO2007072219A2 discloses methods for detecting a neoplasm and/or chemotherapeutic drug resistance or angiogenic potential in neoplastic cells by detecting an increase in the expression of α-enolase in such cells.

WO2016145113A1 discloses compounds targeting enolase enzymes, in particular compounds inhibiting enolase enzymes, and their use in methods of treatment.

WO2014065572A1 discloses a non-substrate analogue having an enolase inhibitory activity, and a pharmaceutical composition for preventing or treating cancer or enolase-associated diseases, containing the same.

However, many of the previously developed compounds targeting ENO1 render the enzyme inactive, e.g. by interacting with the magnesium ion in the active site of ENO1. Inhibition of the enzymatic activity of ENO1, as triggered by a multitude of ENO1-targeting compounds, can, however, be causative for severe side effects. These drawbacks of existing ENO1-targeting compounds reveal a high demand for discovering and/or developing next generation compounds.

In view of the above, there is a continuing need in the art to identify therapeutic compounds with a potential for the treatment of proliferative disorders, such as cancer. The identification of compounds is urgently needed in order to allow the further development of therapies from the laboratory into the clinical practice. There is also an urgent need for enhancing the amount of potentially useful therapeutic compounds, in order to increase the probability of novel and effective treatment options for diseases, for example for proliferative disorders, such as cancer.

Thus, it is an object of the present invention to provide a method for identifying and/or characterizing compounds suitable for the prevention and/or treatment of a disease, for example for the prevention and/or treatment of a proliferative disease, such as cancer. It is a further object of the present invention to control cell differentiation using the identified compounds of the present invention, without risking side effects upon administration of the compounds as identified, to a subject.

### BRIEF DESCRIPTION OF THE INVENTION

Generally, and by way of brief description, the main aspects of the present invention can be described as follows:

In a first aspect, the invention pertains to a method for identifying and/or characterizing a compound suitable for the prevention and/or treatment of a disease.

In a second aspect, the invention pertains to a mutated Enolase 1 (ENO1) enzyme, or a functional fragment thereof.

In a third aspect, the invention pertains to an isolated nucleic acid comprising a sequence coding for the mutated ENO1 enzyme, or a vector comprising the nucleic acid.

In a fourth aspect, the invention pertains to a recombinant cell comprising a mutated ENO1 enzyme, a nucleic acid coding for the mutated ENO1 enzyme, or a vector comprising the nucleic acid.

In a fifth aspect, the invention pertains to a pharmaceutical composition comprising the mutated ENO1 enzyme, the nucleic acid coding for the mutated ENO1 enzyme, the vector comprising the nucleic acid, or the recombinant cell comprising the same.

In a sixth aspect, the invention relates to a method for diagnosing, prognosing, stratifying and/or monitoring of a therapy, of a disease in a subject.

### DETAILED DESCRIPTION OF THE INVENTION

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

In the first aspect, the invention pertains to a method for identifying and/or characterizing a compound suitable for the prevention and/or treatment of a disease, the method comprising the steps of:
a) Providing at least one enzyme of the glycolytic pathway, at least one nucleic acid, and a candidate compound;
b) Bringing into contact the at least one enzyme of the glycolytic pathway, the at least one nucleic acid and the candidate compound;
c) Detecting and/or quantifying a binding between the at least one enzyme of the glycolytic pathway and the at least one nucleic acid;
   wherein a differential level of the binding between the at least one enzyme of the glycolytic pathway and the at least one nucleic acid contacted with the candidate compound compared to the binding between the at least one enzyme of the glycolytic pathway and the at least one nucleic acid not contacted with the candidate compound indicates the candidate compound as suitable for the prevention and/or treatment of the disease.

The inventors surprisingly found that the glycolytic enzyme ENO1 interacts with RNA, and this interaction regulates the glycolytic activity of ENO1. Said riboregulation of ENO1 affects cell differentiation, which plays a pivotal role in cancer. The interaction and/or binding between RNA and ENO1 can, therefore, be used to screen for compounds that can interfere with the binding of RNA to ENO1, which harbors the therapeutic potential for the treatment of diseases, for example proliferative diseases, such as cancer. Hence, the present invention is based on a novel class of compounds that hijack the cells' endogenous riboregulatory mechanisms for therapeutic intervention, and in particular by controlling cell differentiation fate.

The term “at least one” according to the present invention shall include 1, 2, 3, 4, 5, 6, 7, 8, 9, 10, 11, 12, 13, 14, 15, 16, 17, 18, 19, 20, or any other number. For example, the term “at least one” enzyme of the glycolytic pathway shall include 1, 2, 3, 4, 5, 6, 7, 8, 9, 10, 11, 12, 13, 14, 15, 16, 17, 18, 19, 20, or any other number of enzymes of the glycolytic pathway. Similarly, the term “at least one” nucleic acid shall include 1, 2, 3, 4, 5, 6, 7, 8, 9, 10, 11, 12, 13, 14, 15, 16, 17, 18, 19, 20, or any other number of nucleic acids.

In a preferred embodiment, the steps (b) and (c) of the above method for identifying and/or characterizing a compound suitable for the prevention and/or treatment of a disease are performed in a cell-free system, or in a cell, such as in a biological assay cell or in a cell derived from a biological sample, such as a tissue sample or a body liquid sample of a subject, for example a blood sample. In this embodiment, the screening of the invention is performed in a cell-culture system.

The screening of the invention may also be performed in an animal model, for example a mouse or rat cancer model. In said screening method, preferably the use of an animal suffering from a certain disease, such as cancer, is included. The progression of the disease, such as cancer, in said model based on binding of at least one nucleic acid to at least one enzyme of the glycolytic pathway, such as ENO1, of the present invention may be monitored in response to contacting said model with a candidate therapeutic or therapeutic regime. Therefore, a use is preferred wherein in said screening method a test-compound causes a decrease or an increase of the binding of at least one nucleic acid to at least one enzyme of the glycolytic pathway, such as ENO1.

The screening method of the invention is preferably performed in a non-human animal system, ex-vivo, or in-vitro. With respect to ex-vivo uses, the method is preferably done in cell free systems or, alternatively, in cell culture. In context of the latter, mammalian cells are preferably used, in particular human cell lines may be used, such as HeLa cells.

The term “binding” shall refer to any interaction between the at least one enzyme of the glycolytic pathway and the at least one nucleic acid, such as an association between the at least one enzyme of the glycolytic pathway and the at least one nucleic acid. Said association can be direct or indirect. Further included are weak and/or temporary associations between the at least one enzyme of the glycolytic pathway and the at least one nucleic acid

The term “biological sample” as used herein refers to a sample that was obtained and may be assayed for binding between the at least one enzyme of the glycolytic pathway and the at least one nucleic acid, as disclosed in the present invention. The biological sample can include a biological fluid (e.g., blood, cerebrospinal fluid, urine, plasma, serum), tissue biopsy, and the like. In some embodiments, the sample is a tissue sample, such as a tumor tissue sample, and may be fresh, frozen, or archival paraffin embedded tissue. Preferred samples for the purposes of the present invention are bodily fluids, in particular plasma samples.

As used herein, the term “subject” or “patient” refers to any animal (e.g., a mammal), including, but not limited to, humans, non-human primates, rodents, and the like. Typically, the terms “subject” and “patient” are used interchangeably herein in reference to a human subject. As used herein, the term “subject suspected of having a disease” refers to a subject that presents one or more symptoms indicative of a particular disease, such as cancer (e.g., a noticeable lump or mass in the case of cancer). A subject suspected of having a particular disease may also have one or more risk factors. The term a “subject suspected of having a disease” also encompasses an individual who has received an initial diagnosis (e.g., a CT scan showing a mass) but for whom the sub-type or stage of the particular disease, such as a certain type of cancer, is not known. In some embodiments, the subject is an individual who has been recently been diagnosed with the disease. Typically, early treatment (treatment commencing as soon as possible after diagnosis) is important to minimize the effects of the disease and to maximize the benefits of treatment.

Another preferred embodiment relates to the above method for identifying and/or characterizing a compound suitable for the prevention and/or treatment of a disease, wherein the at least one nucleic acid as provided in step (a) of the method is a functional or non-functional RNA polynucleotide molecule or a functional or non-functional DNA polynucleotide molecule, such as a single-stranded or doubled-stranded RNA polynucleotide molecule or DNA polynucleotide molecule, or a fragment or derivative thereof, for example an mRNA molecule, an RNA mimic, an RNA precursor, an RNA analogue, an RNA antisense molecule, an inhibitory RNA molecule, a ribozyme, an RNA antisense expression molecule, an RNA interference (RNAi) molecule, an siRNA molecule, an esiRNA molecule, an shRNA molecule, a miRNA molecule, a DNA mimic, a DNA precursor, a DNA analogue, an antisense DNA, a DNA aptamer, a decoy molecule, a GapmeR, a PNA (peptide nucleic acid) molecule, an LNA molecule (locked nucleic acid), a genetic construct for targeted gene editing, such as a CRISPR/Cas9 construct, a guide nucleic acid (gRNA or gDNA), and/or a tracrRNA.

In general there are no limits to the nature of the candidate compound usable in the screening of the invention. In a particularly preferred embodiment, the candidate compound is selected from a small molecular compound (“small molecule”), a polypeptide, a peptide, a glycoprotein, a peptidomimetic, an antigen binding construct (for example, an antibody, antibody-like molecule or other antigen binding derivative, or an antigen binding fragment thereof), a nucleic acid, such as a DNA or RNA, for example an antisense or inhibitory DNA or RNA, a ribozyme, an RNA or DNA aptamer, RNAi, siRNA, shRNA and the like, including variants or derivatives thereof, such as a peptide nucleic acid (PNA), a genetic construct for targeted gene editing, such as a CRISPR/Cas9 construct, a guide nucleic acid (gRNA or gDNA), and/or a tracrRNA.

The term “small molecule”, as used herein, refers to an organic or inorganic molecule, either synthesized or found in nature. A “small molecule” generally has a molecular weight equal to or less than 1000 Da (1 kDa), however the definition of a small molecule is in some embodiments not limited by this number.

The person of skill is aware of various methods for determining and/or quantifying at least one bi-molecular interaction qualitatively or quantitatively. The present invention shall encompass all such prior art methods applicable by the person skilled in the art. However, in some embodiments, the detecting and/or quantifying in step (c) preferably involves at least one of:
(i) UV cross-linking, immunoprecipitation and radioactive labelling of co-purified RNA (PNK assay);
(ii) Enhanced Crosslinking and Immunoprecipitation (eCLIP);
(iii) Photoactivatable Ribonucleoside-Enhanced Crosslinking and Immuno-precipitation (PAR-CLIP);
(iv) RNA immunopurification followed by microarray hybridization (RIP-chip);
(v) RNA immunopurification followed by high throughput sequencing (RIP-seq);
(vi) RNA-protein crosslink;
(vii) RNA pulldown;
(viii) Mass-spectrometry;
(ix) Proximity Extension Assay;
(x) Immunofluorescent based assays;
(xi) Proximity Ligation Assay;
(xii) Förster resonance energy transfer (FRET), and/or
(xiii) Any other method for reporting at least one bi-molecular interaction.

The present invention shall, however, not be restricted to any particular method for detecting and/or quantifying the binding between the at least one enzyme of the glycolytic pathway and the at least one nucleic acid in step (c) of the screening method of this invention, but shall encompass all means that allow for determination, either directly or indirectly.

The term “differential level” of a binding of the at least one enzyme of the glycolytic pathway to at least one nucleic acid as used herein shall refer to a binding of the at least one enzyme of the glycolytic pathway to at least one nucleic acid that is reduced, enhanced, modified, stabilized, destabilized, mimicked, and/or altered, when compared to a reference or standard value. For example, the term “differential level” of a binding of the at least one enzyme of the glycolytic pathway to at least one nucleic acid can refer to a reduced, enhanced, modified, stabilized, destabilized, mimicked, and/or altered binding of the at least one enzyme of the glycolytic pathway to at least one nucleic acid upon contacting the at least one enzyme of the glycolytic pathway, and the at least one nucleic acid with a compound suitable for the prevention and/or treatment of a disease, when compared to the binding of the at least one enzyme of the glycolytic pathway to at least one nucleic acid not contacted with a compound suitable for the prevention and/or treatment of a disease, or contacted with a candidate compound that is not a compound suitable for the prevention and/or treatment of a disease.

Yet another particularly preferred embodiment relates to the above method, wherein the candidate compound is suitable for the prevention and/or treatment of the disease if the differential level of the binding between the at least one enzyme of the glycolytic pathway and the at least one nucleic acid contacted with the candidate compound compared to the binding between the at least one enzyme of the glycolytic pathway and the at least one nucleic acid not contacted with the candidate compound as detected and/or quantified in step (c) is reduced or enhanced by at least 1%, preferably by at least 2%, more preferably by at least 5%, even more preferably by at least 10%, even more preferably by at least 20%, even more preferably by at least 50%, and most preferably by around 100%.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure. In particularly preferred embodiments of the invention, the term “about” may refer to a deviation of the respective numeric value of a maximum of 20% of the numerical value, however more preferred is 15%,10%, 5%, even more preferred is 4%, 3%, 2%, and most preferred is 1%.

Compositions and methods of the present invention may be used to effectively treat individuals suffering from a disease, such as cancer, or to prevent the onset of a particular disease. As used herein, the term “prevention” shall refer to preventing or delaying the onset of a clinically evident disease, such as a proliferative disease, like cancer, in a subject. The term “treatment”, as used herein, refers to amelioration of one or more symptoms, in particular a partial or total inhibition and/or reduction of symptoms, associated with the disease, such as a proliferative disease, like cancer, in a subject, prevention or delay of the onset of one or more symptoms of the disease, and/or lessening of the severity or frequency of one or more symptoms of the disease. The term “treatment” shall also refer to a partial or total destruction of, for example, diseased cells, or cancer cells. In the context of the present invention, prevention and/or treatment shall include both preventive and/or actual treatment of the disease symptoms of a disease, such as a proliferative disease, like cancer, which can be alleviated and/or even completely removed using said treatment.

In some embodiments, treatment refers to an increased survival (e.g. an increased survival time). For example, treatment can result in an increased life expectancy of a patient. In some embodiments, treatment according to the present invention results in an increased life expectancy of a patient by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, about 200% or more, as compared to the average life expectancy of one or more control individuals with similar disease without treatment. In some embodiments, treatment according to the present invention results in an increased life expectancy of a patient by more than about 6 month, about 7 months, about 8 months, about 9 months, about 10 months, about 11 months, about 12 months, about 2 years, about 3 years, about 4 years, about 5 years, about 6 years, about 7 years, about 8 years, about 9 years, about 10 years or more, as compared to the average life expectancy of one or more control individuals with a similar disease without treatment. In some embodiments, treatment according to the present invention results in long term survival of a patient. As used herein, the term “long term survival” refers to a survival time or life expectancy longer than about 40 years, 45 years, 50 years, 55 years, 60 years, or longer.

The terms, “improve,” “increase” or “reduce,” as used herein, indicate values that are relative to a control. In some embodiments, a suitable control is a baseline measurement, such as a measurement in the same individual prior to initiation of the treatment described herein, or a measurement in a control individual (or multiple control individuals) in the absence of the treatment described herein. A “control individual” is an individual afflicted with the same form of disease, who is about the same age and/or gender as the individual being treated.

In another particularly preferred embodiment of this invention, the at least one enzyme of the glycolytic pathway is Enolase 1 (ENO1), or a derivative, a precursor, a mutant, or a functional fragment thereof, comprising the amino acid sequence according to SEQ ID NO: 1, or an amino acid sequence having at least 80%, preferably at least 90%, more preferably at least 95%, and most preferably at least 99% sequence identity to SEQ ID NO: 1.

In the context of the present invention the term “ENO1” refers to the Enolase 1 enzyme. The term shall encompass the human version of this protein, but also its homologs, in particular in mouse or rat. Information on ENO1 can be derived from the human gene names webpage (https://www.genenames.org/) under the accession number HGNC: 3350. The ENO1 protein is accessible on the UniProt webpage under the accession number P06733, and is further, at least the human version, shown in SEQ ID NO: 1. All other isoforms of the protein shall also be encompassed by the present invention.

Yet another preferred embodiment relates to the above method, wherein the at least one nucleic acid has a length of at least 5 nucleotides, preferably at least 10 nucleotides, more preferably at least 20 nucleotides, even more preferably at least 30 nucleotides, and most preferably at least 35 nucleotides.

In a further preferred embodiment, the at least one nucleic acid comprises at least one modification, for example a chemical modification selected from a modified internucleoside linkage, a modified nucleobase, or a modified sugar moiety, such as a 2′-O-alkyl modification, for example a 2′-O-methoxy-ethyl (MOE) or 2′-O-Methyl (OMe) modification, an ethylene-bridged nucleic acid (ENA), a 2′-fluoro (2′-F) nucleic acid, such as 2′-fluoro N3-P5′-phosphoramidite, a 1′,5′-anhydrohexitol nucleic acid (HNA), or a locked nucleic acid (LNA).

Interestingly, the inventors determined the RNA-binding sites of ENO1 in the transcriptome by applying enhanced crosslinking and immunoprecipitation (eCLIP) of ENO1, which enabled to identify RNA-binding sites on a transcriptome-wide scale, and revealed RNA-binding sites at nucleotide resolution. Based on the exact crosslinking sites as identified by the inventors, approximately two thousand direct ENO1-binding sites across the transcriptome (FIG. 1b) that do not display striking linear sequence motif recognition. The top scoring two sequence motifs (FIG. 2: TTTTTTBTTTTTT, and CCCAGRC) jointly account for ˜22% of all ENO1 binding sites as identified by the inventors.

Another preferred embodiment, therefore, relates to the above method, wherein the at least one nucleic acid comprises the nucleic acid sequence according to SEQ ID NO: 2 (TTTTTTBTTTTTT), or a nucleic acid sequence having at least 80%, preferably at least 90%, more preferably at least 95%, and most preferably at least 99% sequence identity to SEQ ID NO: 2, and/or the nucleic acid sequence according to SEQ ID NO: 3 (CCCAGRC), or a nucleic acid sequence having at least 80%, preferably at least 90%, more preferably at least 95%, and most preferably at least 99% sequence identity to SEQ ID NO: 3.

Approximately two thousand direct ENO1-binding sites across the transcriptome were identified by the inventors using eCLIP of ENO1. Three examples of these approximately two thousand direct ENO1-binding sites are present in PABPC1, PTP4A1 and FTH1 mRNA. In a particularly preferred embodiment, the at least one nucleic acid is, therefore, PABPCi-mRNA, or a precursor, a fragment, or a mutant thereof, comprising a nucleic acid sequence encoding for an amino acid sequence according to SEQ ID NO: 4, or encoding for an amino acid sequence having at least 80%, preferably at least 90%, more preferably at least 95% sequence identity to SEQ ID NO: 4; FTH1-mRNA, or a precursor, a fragment, or a mutant thereof, comprising a nucleic acid sequence encoding for an amino acid sequence according to SEQ ID NO: 5, or encoding for an amino acid sequence having at least 80%, preferably at least 90%, more preferably at least 95% sequence identity to SEQ ID NO: 5; and/or PTP4A1-mRNA, or a precursor, a fragment, or a mutant thereof, comprising a nucleic acid sequence encoding for an amino acid sequence according to SEQ ID NO: 6, or encoding for an amino acid sequence having at least 80%, preferably at least 90%, more preferably at least 95% sequence identity to SEQ ID NO: 6.

The term “encoding” or more simply “coding” refers to the ability of a nucleotide sequence to code for one or more amino acids. The term does not require a start or stop codon. An amino acid sequence can be encoded in any one of six different reading frames provided by a polynucleotide sequence and its complement. An amino acid sequence can be encoded by desoxyribonucleic acid (DNA), ribonucleic acid (RNA), or artificially synthesized polymers similar to DNA or RNA.

In the context of the present invention the term “PABPCi” refers to poly(A) binding protein cytoplasmic 1. The term shall encompass the human version of this protein, but also its homologs, in particular in mouse or rat. Information on PABPC1 can be derived from the human gene names webpage (https://www.genenames.org/) under the accession number HGNC: 8554. The PABPC1 protein is accessible on the UniProt webpage under the accession number P11940, and is further, at least the human version, shown in SEQ ID NO: 4. All other isoforms of the protein shall also be encompassed by the present invention.

Further, as used herein, the term “FTH1” refers to Ferritin heavy chain 1 (FTH1). The term shall encompass the human version of this protein, but also its homologs, in particular in mouse or rat. Information on FTH1 can be derived from the human gene names webpage (https://www.genenames.org/) under the accession number HGNC: 3976. The FTH1 protein is accessible on the UniProt webpage under the accession number P02794, and is further, at least the human version, shown in SEQ ID NO: 5. All other isoforms of the protein shall also be encompassed by the present invention.

In the context of the present invention the term “PTP4A1” refers to poly(A) binding protein cytoplasmic 1. The term shall encompass the human version of this protein, but also its homologs, in particular in mouse or rat. Information on PTP4A1 can be derived from the human gene names webpage (https://www.genenames.org/) under the accession number HGNC: 9634. The PTP4A1 protein is accessible on the UniProt webpage under the accession number Q93096, and is further, at least the human version, shown in SEQ ID NO: 6. All other isoforms of the protein shall also be encompassed by the present invention.

All references to the databases in context of the invention refer to their respective versions of Sep. 29, 2020.

The general idea of the invention is the use of the method in the production and identification of therapeutics, such as anti-cancer therapeutics. According to the present invention, any disease, condition or disorder can be prevented and/or treated by the compound as identified and/or characterized according to the method of this invention. In a preferred embodiment, said disease is a proliferative disease, such as cancer, diabetes, an infectious disease, a metabolic disease, an immune-related disease, a degenerative disease, such as a neurodegenerative disease, for example Alzheimer's disease, and/or aging.

The term “cancer” and “cancer cells” refers to any cells that exhibit uncontrolled growth in a tissue or organ of a multicellular organism. The term “cancer” is understood to mean any cancer or cancerous lesion associated with a certain tissue or tissue cells and can include precursors to the particular cancer disease, for example, atypical ductal hyperplasia or non-atypical hyperplasia. Cancer can be understood as a disease in which a primary tumor or multiple individual primary tumors exist, e.g. in the breast or breasts in case of breast cancer. The term “tumor” refers to an abnormal benign or malignant mass of tissue that is not inflammatory and possesses no physiological function.

Further preferred is that the compound identified and/or characterized by the method of this invention is suitable for the prevention and/or treatment of a disease, wherein the disease is characterized by an altered level of glycolysis in at least one cell compared to a control or reference value or cell, such as an enhanced or a reduced level of glycolysis in the at least one cell compared to the control or reference value or cell, and wherein said candidate compound reduces or enhances the altered level of glycolysis in the at least one cell.

Yet another preferred embodiment relates to the above method, wherein the candidate compound regulates the enzymatic activity of the at least one enzyme of the glycolytic pathway. Importantly, the inventors' biochemical studies and the data using the ENO1 mutants support that RNA directly interacts with the enzyme in a way that is mutually exclusive with substrate binding, which explains the riboregulation of ENO1.

A further aspect of this invention relates to a diagnostic kit for performing a method for identifying and/or characterizing a compound suitable for the prevention and/or treatment of a disease as defined above, wherein the kit comprises at least one enzyme of the glycolytic pathway, and optionally at least one nucleic acid. The above kit can further include an agent for detection of the at least one enzyme of the glycolytic pathway, the at least one nucleic acid, and/or the binding between the at least one enzyme of the glycolytic pathway and the at least one nucleic acid, such as an antigen binding construct (for example, an antibody, an antibody-like molecule or other antigen binding derivative, or an antigen binding fragment thereof), a nucleic acid, including an RNA or DNA aptamer, and the like. Further, instructions for use can be included in the diagnostic kit.

A “diagnosis” or the term “diagnostic” in the context of the present invention means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

Yet another aspect of this invention, which can be combined with any of the other preferred embodiments and/or aspects of the invention, pertains to the use of at least one enzyme of the glycolytic pathway, in the performance of a method for identifying and/or characterizing a compound suitable for the prevention and/or treatment of a disease, as defined above.

A further aspect of this invention, which can be combined with any of the other preferred embodiments and/or aspects of the invention, relates to a method for the production of a pharmaceutical composition, the method comprising identifying a compound with a method for identifying and/or characterizing a compound suitable for the prevention and/or treatment of a disease, according to this invention and as defined above, and formulating the compound as a pharmaceutical composition, together with a pharmaceutically acceptable carrier and/or excipient.

Another aspect of this invention, which can be combined with any of the other preferred embodiments and/or aspects of the invention, pertains to the use of a compound identified according to the method for identifying and/or characterizing a compound suitable for the prevention and/or treatment of a disease, as defined above, for the production of a medicament for use in the prevention and/or treatment of a disease.

Yet another aspect of this invention, which can be combined with any of the other preferred embodiments and/or aspects of the invention, pertains to a mutated Enolase 1 (ENO1) enzyme, or a functional fragment thereof, wherein the mutated ENO1 enzyme amino acid sequence when aligned to the amino acid sequence of SEQ ID NO: 1 comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to SEQ ID NO: 1.

The term “mutated” and/or “mutation” refers to, in the context of a polynucleotide, a modification to the polynucleotide sequence resulting in a change in the sequence of a polynucleotide with reference to a precursor polynucleotide sequence. A mutant polynucleotide sequence can refer to an alteration that does not change the encoded amino acid sequence, for example, with regard to codon optimization for expression purposes, or that modifies a codon in such a way as to result in a modification of the encoded amino acid sequence. Mutations can be introduced into a polynucleotide through any number of methods known to those of ordinary skill in the art, including random mutagenesis, site-specific mutagenesis, oligonucleotide directed mutagenesis, gene shuffling, directed evolution techniques, combinatorial mutagenesis, site saturation mutagenesis among others.

A particularly preferred embodiment of the present invention pertains to a mutated ENO1 enzyme, or the functional fragment thereof, as defined above, comprising an amino acid sequence with at least 80%, preferably at least 90%, more preferably at least 95%, and most preferably at least 99% sequence identity to SEQ ID NO: 1.

Another preferred embodiment of the present invention pertains to a mutated ENO1 enzyme, or the functional fragment thereof, as defined above, wherein the amino acid sequence of the mutated ENO1 enzyme, or of the functional fragment thereof, when aligned to the amino acid sequence of SEQ ID NO: 1, comprises at least one amino acid substitution, deletion, and/or addition in an amino acid at positions 57-132 of SEQ ID NO: 1, or at position 343 of SEQ ID NO: 1.

A particularly preferred embodiment relates to the mutated ENO1 enzyme, or the functional fragment thereof, wherein the amino acid sequence of the mutated ENO1 enzyme, or of the functional fragment thereof, comprises at least one amino acid substitution, deletion, and/or addition at position 89, 92, 105, and/or 343 of SEQ ID NO: 1, and preferably comprises the amino acid sequence of any of SEQ ID NOs: 7 to 14, or comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to the sequence of any of SEQ ID NOs: 7 to 14.

Another preferred embodiment relates to the mutated ENO1 enzyme, or the functional fragment thereof, comprising at least one amino acid substitution selected from K89A, K92A, and K105A at positions 89, 92, and 105 in SEQ ID NO: 1, or comprising the amino acid substitutions K89A and K92A at positions 89, and 92 in SEQ ID NO: 1, or comprising the amino acid substitutions K89A and K105A at positions 89, and 105 in SEQ ID NO: 1, or comprising the amino acid substitutions K92A and K105A at positions 92, and 105 in SEQ ID NO: 1, or comprising the amino acid substitutions K89A, K92A, and K105A at positions 89, 92, and 105 in SEQ ID NO: 1, wherein said mutated ENO1 enzyme is characterized by an enhanced binding of at least one nucleic acid to the mutated ENO1 enzyme compared to the binding of the at least one nucleic acid to the wild type ENO1 enzyme comprising the amino acid sequence of SEQ ID NO: 1, preferably wherein the mutated ENO1 enzyme comprises the amino acid sequence of any of SEQ ID NOs: 7 to 13, or comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to the sequence of any of SEQ ID NOs: 7 to 13.

Interestingly, the inventors successfully generated a mutant of ENO1, referred to herein as “ENO1up mutant”. The design of the ENO1up mutant was guided by RBDmap data and entails the change of lysine residues (K89A/K92A/K105A) along the inferred interaction region of RNA with the enzyme. After knocking down endogenous HeLa cell ENO1 and rescue with the respective Flag-tagged ENO1 variant, ENO1up displays increased RNA binding compared to ENO1wt, as measured by PNK assays (FIGS. 7a and b). When the inventors tested the ENO1up mutant for its ability to rescue glycolysis (lactate accumulation in the medium) in HeLa cells after knock-down of endogenous ENO1, ENO1up fails to rescue the knock-down-induced inhibition of lactate accumulation (FIG. 7b), although it is fully active when tested in the absence of RNA in vitro (FIGS. 7c and d, FIG. 8a). This supports the finding that ENO1's enzymatic substrate binding and RNA binding are competitive.

Yet another preferred embodiment relates to the mutated ENO1 enzyme, or the functional fragment thereof, comprising at least one amino acid substitution at position 343 in SEQ ID NO: 1, such as a K343A amino acid substitution at position 343 in SEQ ID NO: 1, wherein said mutated ENO1 enzyme is characterized by a reduced binding of at least one nucleic acid to the mutated ENO1 enzyme compared to the binding of the at least one nucleic acid to the wild type ENO1 enzyme comprising the amino acid sequence of SEQ ID NO: 1, preferably wherein the mutated ENO1 enzyme comprises the amino acid sequence of SEQ ID NO: 14, or comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to SEQ ID NO: 14.

Instructed by RBDmap data, the inventors generated an ENO1 mutant (K343A; referred to herein as “ENO1down”) with ˜5-10-fold decreased RNA binding (FIGS. 5c and d, FIG. 6) compared to ENO1wt as measured by competitive EMSA (FIG. 4, FIGS. 5c and d). Of note, the RNA binding-deficient mutant ENO1down is as enzymatically active as the wild-type protein, showing that the K343A mutation does not incapacitate the enzyme. Interestingly, ENO1down displays substantially decreased RNA binding in HeLa cells relative to ENO1wt (FIG. 7a). The inventors surmise that ENO1down is RNA-binding deficient because K343 directly interacts with RNA.

Also preferred is the mutated ENO1 enzyme, or the functional fragment thereof, as defined above, wherein the mutated ENO1 enzyme, or the functional fragment thereof, has a comparable enzymatic activity as wild type ENO1 comprising SEQ ID NO: 1. Nevertheless, mutated ENO1 enzymes, or functional fragments thereof, shall also be within the scope of the present invention, wherein the mutated ENO1 enzyme, or the functional fragment thereof, has an increased and/or decreased enzymatic activity compared to the enzymatic activity of wild type ENO1 comprising SEQ ID NO: 1.

The person of skill is aware of methods for analyzing the enzymatic activity of an enzyme, such as ENO1, e.g. by performing an enzymatic assay of ENO1. According to this invention, the term “determining the activity” or “determining the enzymatic activity” can further include analyzing post-translational modifications of the particular enzyme, such as ENO1, wherein the activity of the enzyme is dependent upon at least one of the post-translational modifications of an amino acid of the particular enzyme. How to determine post-translational modifications of an enzyme and/or protein is well known to the skilled artisan, and may, in one particular embodiment, involve western blotting or ELISA. The present invention shall, however, not be restricted to any particular method for determining the enzymatic activity, but shall encompass all means that allow for determination, either directly or indirectly.

Yet another aspect of this invention, which can be combined with any of the other preferred embodiments and/or aspects of the invention, pertains to an isolated nucleic acid comprising a sequence coding for the mutated ENO1 enzyme, or the functional fragment thereof, as defined above.

The term “expression construct” means any double-stranded DNA or double-stranded RNA designed to transcribe an RNA, e.g., a construct that contains at least one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus, or further embodiments described under “expression vector” below. An expression construct can be replicated in a living cell, or it can be made synthetically. For purposes of this application, the terms “expression construct”, “expression vector”, “vector”, and “plasmid” are used interchangeably to demonstrate the application of the invention in a general, illustrative sense, and are not intended to limit the invention to a particular type of expression construct. Further, the term expression construct or vector is intended to also include instances wherein the cell utilized for the assay already endogenously comprises such DNA sequence.

Another aspect of the invention provides a vector, comprising the nucleic acid of the invention. In a preferred embodiment, the vector comprising the nucleic acid is an expression vector, comprising a promoter sequence operably linked to the nucleic acid as defined above.

A “vector” may be any agent that is able to deliver or maintain a nucleic acid in a host cell and includes, for example, but is not limited to, plasmids (e.g., DNA plasmids), naked nucleic acids, viral vectors, viruses, nucleic acids complexed with one or more polypeptide or other molecules, as well as nucleic acids immobilized onto solid phase particles. Vectors are described in detail below. A vector can be useful as an agent for delivering or maintaining an exogenous gene and/or protein in a host cell. A vector may be capable of transducing, transfecting, or transforming a cell, thereby causing the cell to replicate or express nucleic acids and/or proteins other than those native to the cell or in a manner not native to the cell. The target cell may be a cell maintained under cell culture conditions or in other in vivo embodiments, being part of a living organism. A vector may include materials to aid in achieving entry of a nucleic acid into the cell, such as a viral particle, liposome, protein coating, or the like. Any method of transferring a nucleic acid into the cell may be used; unless otherwise indicated, the term vector does not imply any particular method of delivering a nucleic acid into a cell or imply that any particular cell type is the subject of transduction. The present invention is not limited to any specific vector for delivery or maintenance of any nucleic acid of the invention, including, e.g., a nucleic acid encoding a mutant ENO1 polypeptide of the invention or a fragment thereof.

In another aspect there is also provided a recombinant cell comprising a mutated ENO1 enzyme, or the functional fragment thereof, a nucleic acid, or a vector of the invention as described herein. A “recombinant cell”, sometimes also referred to as “host cell”, is any cell that is susceptible to transformation with a nucleic acid. Preferably, the recombinant or host cell of the invention is a plant cell, bacterial cell, yeast cell, an insect cell or a mammalian cell. A preferred recombinant cell is selected from a cell suitable for recombinant expression of the mutated ENO1 of the invention. Also preferred are human cells, preferably autologous human cells derived from a patient suffering from a disease described herein that is treatable with a mutated ENO1 of the invention.

In another aspect there is provided a pharmaceutical composition comprising a mutated ENO1 enzyme, or the functional fragment thereof, a nucleic acid, a vector, or a recombinant cell of the invention as described before, together with a pharmaceutically acceptable carrier, stabilizer and/or excipient.

In the following, the mutated ENO1 enzyme, or the functional fragment thereof, the, nucleic acids encoding the same, vectors and cells comprising these nucleic acids or mutated proteins, as well as pharmaceutical compositions thereof, will be referred to generally as “compounds of the invention”.

The mutated ENO1 enzyme, or the functional fragment thereof, of the invention is in some preferred embodiments an isolated ENO1 enzyme or a recombinant ENO1 enzyme. The term “recombinant” or “recombinantly produced” in context of the invention means that a protein or peptide is expressed via an artificially introduced exogenous nucleic acid sequence in a biological cell. Recombinant expression is usually performed by using expression vectors as described herein elsewhere.

An additional aspect of this invention pertains to a pharmaceutical composition comprising the mutated ENO1 enzyme, or the functional fragment thereof, the nucleic acid, the vector, or the recombinant cell as defined above, together with a pharmaceutically acceptable carrier, stabilizer and/or excipient.

As used herein the term “pharmaceutically acceptable carrier” shall include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Supplementary agents can also be incorporated into the compositions.

The pharmaceutical composition of the invention is formulated to be compatible with its intended route of administration. In a particularly preferred embodiment examples of routes of administration of the pharmaceutical and/or compound of this invention include intravenous, vaginal, oral, intranasal, intrathecal, intra-arterial, intradermal, subcutaneous, transdermal (topical), intracerebroventricular, intraparenchymal, intratumoral, transmucosal, rectal, bronchial, parenteral administration, and any other clinically/medically accepted method for administration of a pharmaceutical and/or a compound.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride, mannitol or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, mannitol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

The term “intrathecal,” as used herein, means introduced into or occurring in the space under the arachnoid membrane which covers the brain and spinal cord. The term “intracerebroventricular” refers to administration of a composition into the ventricular system of the brain, e.g., via injection, infusion, or implantation (for example, into a ventricle of the brain). As used herein, the term “intraparenchymal” can refer to an administration directly to brain tissue. In other instances, intraparenchymal administration may be directed to any brain region where delivery of one or more compounds of the invention is effective to mitigate or prevent one or more of disorders as described herein.

Sterile injectable solutions can be prepared by incorporating the active compound (e.g., a compound of the invention such as a mutated ENO1) in the required amount in an appropriate solvent with one or a combination of ingredients enumerated above, as required, followed by filtered sterilization. Generally, dispersions are prepared by incorporating the active compound of the invention into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

A further aspect of this invention relates to a compound for use in the prevention and/or treatment of a disease, the compound being selected from a mutated ENO1 enzyme, or the functional fragment thereof, the nucleic acid, the vector, the recombinant cell, and a pharmaceutical composition according as defined above, together with a pharmaceutically acceptable carrier, stabilizer and/or excipient.

Yet another aspect of this invention, which can be combined with any of the other aspects or specific embodiments of this invention, relates to a method of preventing and/or treating a disease, condition or disorder in a patient, comprising the administration to the subject a therapeutically effective amount of a compound according to this invention.

A treatment according to the invention preferably comprises the administration of a therapeutically effective amount of the compound of the invention to a subject in need of the treatment. The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing and/or treating a target condition, alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser, together with instructions for administration.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, serum albumin, polyorthoesters, polylactic acid, poly(butyl cyanoacrylate), and poly(lactic-co-glycolic) acid. Methods for preparation of such formulations will be apparent to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

An additionally preferred embodiment of the invention pertains to the above method of treatment, wherein said treatment comprises administration to said subject a compound as screened according to the method for identifying and/or characterizing a compound suitable for the prevention and/or treatment of a disease, as defined above.

Another aspect of this invention, which can be combined with any of the other aspects or specific embodiments of this invention, relates to the use of a compound according to this invention, for the manufacture of a medicament for the prevention and/or treatment of a disease, condition or disorder.

Yet another aspect of this invention pertains to a method for identifying a mutated enzyme of the glycolytic pathway for use in a method for identifying and/or characterizing a compound suitable for the prevention and/or treatment of a disease, as defined above, the method comprising the steps of:
a) Providing at least one wild type enzyme of the glycolytic pathway, at least one mutated enzyme of the glycolytic pathway, and at least one nucleic acid;
b) Bringing into contact the at least one wild type enzyme of the glycolytic pathway, and the at least one nucleic acid;
c) Bringing into contact the at least one mutated enzyme of the glycolytic pathway, and the at least one nucleic acid;
d) Detecting and/or quantifying a binding between the at least one wild type enzyme of the glycolytic pathway and the at least one nucleic acid, and detecting and/or quantifying the binding between the at least one mutated enzyme of the glycolytic pathway and the at least one nucleic acid,
   wherein a differential level of the binding between the at least one wild type enzyme of the glycolytic pathway and the at least one nucleic acid compared to the binding between the at least one mutated enzyme of the glycolytic pathway and the at least one nucleic acid indicates the mutated enzyme of the glycolytic pathway as suitable for use in a method for identifying and/or characterizing a compound suitable for the prevention and/or treatment of a disease as defined herein above.

A preferred embodiment relates to the above method for identifying a mutated enzyme of the glycolytic pathway, wherein said differential level of the binding between the at least one wild type enzyme of the glycolytic pathway and the at least one nucleic acid compared to the binding between the at least one mutated enzyme of the glycolytic pathway and the at least one nucleic acid is an enhanced or a reduced binding between the at least one wild type enzyme of the glycolytic pathway and the at least one nucleic acid compared to the binding between the at least one mutated enzyme of the glycolytic pathway.

In all aspects and embodiments of the present invention it may be preferred that the at least one wild type enzyme of the glycolytic pathway is Enolase 1 (ENO1) comprising the amino acid sequence of SEQ ID NO: 1, and wherein the at least one mutated enzyme of the glycolytic pathway is a derivative, a precursor, a mutant, or a functional fragment of ENO1, comprising not more than 50 amino acid substitutions, deletions, and/or additions of the amino acid sequence according to SEQ ID NO: 1, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of the amino acid according to SEQ ID NO: 1.

A particularly preferred embodiment relates to the above method for identifying a mutated enzyme of the glycolytic pathway, wherein the at least one mutated enzyme of the glycolytic pathway comprises an amino acid sequence with at least 80%, preferably at least 90%, more preferably at least 95%, and most preferably at least 99% sequence identity to SEQ ID NO: 1.

As used herein, the terms “identical” or percent “identity”, when used anywhere herein in the context of two or more nucleic acid or protein/polypeptide sequences, refer to two or more sequences or subsequences that are the same or have (or have at least) a specified percentage of amino acid residues or nucleotides that are the same (i.e., at, or at least, about 60% identity, preferably at, or at least, 65%, 70%, 75%, 8o%, 85%, 90%, 91%, 92%, 93% or 94%, identity, and more preferably at, or at least, about 95%, 96%, 97%, 98%, 99%, or higher identity over a specified region—preferably over their full length sequences—, when compared and aligned for maximum correspondence over the comparison window or designated region) as measured using a sequence comparison algorithms, or by manual alignment and visual inspection (see, e.g., NCBI web site). In a particular embodiment, for example when comparing the protein or nucleic acid sequence of a mutated ENO1 with wild-type ENO1, the percentage identity can be determined by the Blast searches or local alignments.

A further aspect of this invention, which can be combined with any of the other preferred embodiments and/or aspects of this invention, pertains to a method for identifying and/or characterizing a compound suitable for the prevention and/or treatment of a disease, the method comprising the steps of:
a) Providing at least one enzyme of the glycolytic pathway, and a candidate compound;
b) Bringing into contact the at least one enzyme of the glycolytic pathway, and the candidate compound
c) Detecting and/or quantifying at least one modification in the at least one enzyme of the glycolytic pathway;
   wherein a differential level of the at least one modification in the at least one enzyme of the glycolytic pathway contacted with the candidate compound compared to the at least one modification in the at least one enzyme of the glycolytic pathway not contacted with the candidate compound indicates the candidate compound as suitable for the prevention and/or treatment of the disease, and wherein the differential level of the at least one modification is indicative for a differential level of a binding of the at least one enzyme of the glycolytic pathway to at least one nucleic acid.

Interestingly, the data provided by the inventors disclose that riboregulation of ENO1 is (at least partially) controlled by at least one posttranslational modification of ENO1. Said posttranslational modification, such as ubiquitination, controls the overall ability of ENO1 to bind RNA. Accordingly, at least one posttranslational modification of ENO1, such as ubiquitination, represents an attractive possibility for controlling riboregulation of ENO1, and making use of said mechanism when developing treatment strategies. Interestingly, the ENO1up mutant (ENO1i-K89A/K92A/K105A mutant) developed by the inventors suggests candidate sites for such a modification, i.e. at least one ubiquitination, at location K89, K92, and/or K105, when aligned to the sequence of human ENO1 according to SEQ ID NO: 1. Importantly, the transcriptome as a whole bearing thousands of relevant ENO1-binding regions serves a very specific regulatory function.

In a preferred embodiment, the modification as detected and/or quantified in the method for identifying and/or characterizing a compound suitable for the prevention and/or treatment of a disease, is selected from ubiquitination, acetylation, phosphorylation, methylation, glycosylation, lipid-conjugation, functionalization, heterodimerization, homodimerization, oxidation, hydroxylation, or any other natural or artificial post-translational modification, or combinations thereof, preferably wherein said modification is a modification of at least one amino acid residue of the at least one enzyme of the glycolytic pathway. In a particularly preferred embodiment, the modification as detected and/or quantified in the method for identifying and/or characterizing a compound suitable for the prevention and/or treatment of a disease, is acetylation.

Another preferred embodiment pertains to the method for identifying and/or characterizing a compound suitable for the prevention and/or treatment of a disease, wherein the at least one enzyme of the glycolytic pathway is Enolase 1 (ENO1), or a derivative, a precursor, a mutant, or a functional fragment thereof, comprising the amino acid sequence according to SEQ ID NO: 1, or an amino acid sequence having at least 80%, preferably at least 90%, more preferably at least 95%, and most preferably at least 99% sequence identity to SEQ ID NO: 1

Yet another aspect of this invention relates to a method for diagnosing, prognosing, stratifying and/or monitoring of a therapy, of a disease in a subject, comprising the steps of:
a) Providing a sample comprising at least one enzyme of the glycolytic pathway from the subject, and at least one nucleic acid, and optionally at least one agent for detection of the at least one enzyme of the glycolytic pathway, the at least one nucleic acid, and/or a binding between the at least one enzyme of the glycolytic pathway and the at least one nucleic acid, such as an antigen binding construct (for example, an antibody, an antibody-like molecule or other antigen binding derivative, or an antigen binding fragment thereof), a nucleic acid, including an RNA or DNA aptamer, and the like;
b) Optionally, isolating the at least one enzyme of the glycolytic pathway from the sample;
c) Bringing into contact the at least one enzyme of the glycolytic pathway, and the at least one nucleic acid, and
d) Detecting and/or quantifying the binding between the at least one enzyme of the glycolytic pathway and the at least one nucleic acid;
   wherein a differential level of the binding between the at least one enzyme of the glycolytic pathway and the at least one nucleic acid in the sample from the subject as detected and/or quantified in step (d) compared to a control or reference value is indicative for the diagnosis, prognosis, stratification and/or monitoring of a therapy, of the disease in the subject.

In a preferred embodiment, the sample as provided in step a) of the above method is a tissue sample or a body liquid sample, such as a blood sample.

A further preferred embodiment relates to the method for diagnosing, prognosing, stratifying and/or monitoring of a therapy, of a disease in a subject, wherein the disease is a proliferative disease, such as cancer, diabetes, an infectious disease, a metabolic disease, an immune-related disease, a degenerative disease, such as a neurodegenerative disease, for example Alzheimer's disease, and/or aging.

A “diagnosis” or the term “diagnosing” or “diagnostic” in the context of the present invention means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The term “prognosis” refers to a forecast as to the probable outcome of the disease as well as the prospect of recovery from the disease as indicated by the nature and symptoms of the case. Accordingly, a negative or poor prognosis is defined by a lower post-treatment survival term or survival rate. Conversely, a positive or good prognosis is defined by an elevated post-treatment survival term or survival rate. Usually, prognosis is provided as the time of progression free survival (PFS) or overall survival (OS). Progression-free survival (PFS), as used in the context of this invention, shall be defined as the length of time during and after the treatment of a disease, such as cancer, that a patient lives with the disease but the disease does not get worse, i.e. survival without progression of the disease. PFS shall further be defined as the time from random assignment, e.g. in a clinical trial, to disease progression or death from any cause. In a clinical trial, measuring the PFS is one way to see how well a new treatment works. The term “overall survival” (OS) as used in the context of this invention shall be defined as the duration of time from the date of diagnosis or commencement of a treatment of a particular disease that a patient is still alive. Thus, in a clinical trial the measure of overall survival would compare the number of patients who had died and the number who had not died.

The term “monitoring of a therapy, of a disease” shall, in the context of the present invention, refer to observing disease progression in a patient who receives a therapy. In other words, the patient during the therapy is regularly monitored for the effect of the applied therapy, which allows the medical practitioner to estimate at an early stage during the therapy whether the prescribed treatment is effective or not, and, therefore, to adjust the treatment regime accordingly.

The term “stratifying” and/or “stratification” for the purposes of this invention shall refer to the advantage that the method according to the invention renders decisions for the treatment and therapy of the patient possible, whether it is the hospitalization of the patient, the use, effect and/or dosage of one or more drugs, a therapeutic measure or the monitoring of a course of the disease and the course of therapy or etiology or classification of a disease, e.g., into a new or existing subtype or the differentiation of diseases and the patients thereof. Particularly with regard to cancer, “stratification” means in this context a classification of a cancer disease of an individual patient with regard of the metastatic status, or the presence or absence of circulating tumor cells. Also, the term “stratification” covers, in particular embodiment, the risk stratification with the prognosis of an outcome of a negative health event.

The above method for diagnosing, prognosing, stratifying and/or monitoring of a therapy, of a disease in a subject, is particularly useful for tumor staging of patients based on detecting and/or quantifying the interaction of RNA with ENO1 in tissue derived from said patient. Such staging enables diagnosing a certain subtype of a disease such as a particular cancer type, prognosing the survival chances of the patient, stratifying patients into different groups, e.g. those that might benefit from a particular treatment and others that might not benefit from the same treatment, and/or monitoring the success of a therapy for the treatment of the disease.

The terms “of the [present] invention”, “in accordance with the invention”, “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### BRIEF DESCRIPTION OF THE FIGURES AND SEQUENCES

The figures show:

FIG. 1 shows that the glycolytic enzyme Enolase 1 (ENO1) binds RNA in vivo. a. ENO1 immunoprecipitation (IP) after crosslinking RNA-RBP complexes with UV light. Polynucleotide kinase labels RNA with radioactive ³²P-γATP (PNK assay). Western blot staining for ENO1 and LARP1 (RNA-binding protein) of the same experiment. Three biological replicates are shown for ENO1 and an IgG control of the same species. b. Volcano plot of differential crosslinking site occurrences as determined by DEWseq; each dot corresponds to a window of genomic region (50 nts), grey colouring indicates significant enrichment in ENO1 IPs (IHW-adjusted p-value <0.1, loge FC>0.5). The data are based on five biological experiments and were normalized to the background. Only the positive enrichment is displayed. c. Top: HeLa cell lysates were treated with RNase I/A/T1 or left untreated, subjected to sucrose density gradient (5-25%), centrifugation and fractionation. A protein's RNase-sensitive fraction moves from the higher fraction number (heavy) to the lower fraction number (light). Bottom: Quantification of biological replicates for the experimental setup on the top for ENO1 immunoblots (n=3). The standard deviation is given.

FIG. 2 shows ENO1's specific RNA binding in HeLa cells. ENO1's binding profile as identified by eCLIP relative to the input control is used for the identification of target and control sites. Top: Enriched ENO1-binding sites are normalised by the length of the feature. Bottom: CL motif analysis based on DEWseq results of ENO1 eCLIP (n=5). Logo representation of the top scoring motifs using DREME. The top scoring two sequence motifs (FIG. 2: TTTTTTBTTTTTT, and CCCAGRC) jointly account for ˜22% of all ENO1 binding sites.

FIG. 3 shows ENO1's specific RNA binding in vitro. a. Schematic of ENO1's binding profile as identified by eCLIP relative to the input control used for the identification of target and control sites. Every line represents an accumulation of crosslink sites at an individual nucleotide. b. Overlay of1H,15N-HSQC spectra of free ENO1 and ENO1 incubated with twofold excess of RNA (FTH1 18-mer start, black). Full titration points (in 1:0.1/0.2/0.4/0.8/1.2/2 ratios) for some residues are shown in insets. c. Comparative electromobility shift assay (EMSA) for target versus control RNA using radioactively labelled PABPC1 target 35-mer as a probe and unlabelled competitor RNA from either the target or control region as indicated in a. d. Inhibition constants (Ki) for target and control RNAs for the binding to ENO1wt (n=3). The Ki was calculated using a non-linear fitting with least squares regression.

FIG. 4 shows in vitro analyses of ENO1 RNA binding specificity by competitive EMSAs. a. Quantification of competition EMSA experiments using FTH1 target RNA as a probe and different unlabelled 18-mer FTH1 RNA competitors (n=3). b. Quantification of competition EMSA experiments using PABPC1 target RNA as a probe and unlabelled PABPC1 control or target RNA competing for the binding to ENO1 (n=3). c. Quantification of competition EMSA experiments using FTH1 target RNA as a probe and unlabelled control or target FTH1 RNA as competitors (n=3). d. Quantification of competition EMSA experiments using PTP4A1 target RNA as a probe and unlabelled control or target (n=3).

FIG. 5 shows riboregulation of ENO1's enzymatic activity in vitro. a. Enzymatic activity assay of recombinant human ENO1 with increasing enzyme concentrations exposed to different control and target RNAs (constant conc. 100 nM). b. Measurement of recombinant ENO1wt activity with increasing concentrations of control and target PABPC1 RNA (n=3). The standard deviation is given and the statistically significant differences were detected using two-way ANOVA and Sidak-correction for multiple comparison testing. c. Inhibition constants (Ki) for target and control RNAs for the binding to ENO1down determined by competitive EMSA (n=3). The Ki was calculated using a non-linear fitting with least squares regression. The respective curves are shown in FIGS. 6a-c. d. Measurement of recombinant ENO1down activity with increasing concentrations of control and target PABPC1 RNA (n=3). e. EMSA with recombinant human ENO1 and a labelled PABPC1 target RNA probe in competition with substrate of the forward (2-phosphoglycerate) and reverse reaction (phosphoenol pyruvate) or a control glycolytic metabolite (3-phosphoglycerate). f. Quantification of replicates for the experimental setup in e, including the inhibitor constants (n=3). The Ki was calculated using a non-linear fitting with least squares regression.

FIG. 6 shows the effect of RNA on the enzymatic activity of ENO1down in vitro. a. Quantification of competition EMSA experiments using PABPC1 target RNA as a probe and unlabelled PABPC1 control or target RNA competing for the binding to ENO1down (n=3). b. Quantification of competition EMSA experiments of ENO1down using FTH1 target RNA as a probe and unlabelled control or target FTH1 RNA as competitors (n=3). c. Quantification of competition EMSA experiments of ENO1down using PTP4A1 target RNA as a probe and unlabelled control or target (n=3). d. Measurement of recombinant ENO1down enzymatic activity with increasing concentrations of control and target FTH1 RNA (n=3). The standard deviation is given and the statistically significant differences were detected using two-way ANOVA and Sidak-correction for multiple comparison testing. e. Experimental setup as in f for control and target PTP4A1 RNA.

FIG. 7 shows Riboregulation of ENO1 in HeLa cells. A. Immunoprecipitation (anti-FLAG) and PNK assay of transiently expressed ENO1-Flag-HA proteins. RNA binding of tagged wild-type ENO1 (ENO1wt), a mutant with increased RNA binding (ENO1up) and reduced RNA binding (ENO1down) are being compared. B. Representative image of a proximity ligation assay (RNA-PLA) for ENO1wt with its mRNA target FTH1. ENO1 is being detected using an anti-FLAG antibody and the endogenous ENO1 is knocked down using siRNAs. The PLA signal is presented as a maximal projection of a Z-stack (10 pictures for 10 μm stack). Pictures for the DAPI (nuclear staining) and cellular outline (grey) are taken in one plane. The scale bar is equivalent to 20 μm. C. Representative image of an RNA-PLA for ENO1down with its mRNA target FTH1. D. Representative image of an RNA-PLA for ENO1up with its mRNA target FTH1. E. Quantification of RNA-PLA signals as dots per cell from three biological replicates with at least 30 cells in total. Statistically significant differences were detected using one-way ANOVA and Tukey-correction for multiple comparison testing. F. Immunofluorescent staining for ENO1wt, ENO1down and ENO1up in transfected HeLa cells using an anti-FLAG antibody, an anti-ENO1 antibody and DAPI. The scale bar is equivalent to 20 μm. G. Representative image of a control RNA-PLA for ENO1wt without the addition of a biotinylated probe. ENO1 is being detected using an anti-FLAG antibody and the endogenous ENO1 is knocked down using siRNAs. The RNA-PLA signal is presented as a maximal projection of a Z-stack (10 pictures for 10 μm stack). Pictures for the DAPI (nuclear staining) and cellular outline (grey) are taken in one plane. The scale bar is equivalent to 20 μm. H. Quantification of RNA-PLA signal of endogenous ENO1 with FTH1 mRNA as dots per cell from three replicates with at least 30 cells in total, comparing samples without probe, cells treated with control siRNA and ENO1 siRNA. Statistically significant differences were detected using one-way ANOVA and Tukey-correction for multiple comparison testing.

FIG. 8 shows the behaviour of ENO1 mutants in HeLa cells and riboregulation in mESCs. a. Comparison of ENO1's RNA-binding (PNK), and ENO1's enzymatic activity in HeLa cells, indirectly measured by the accumulation of lactate in the medium (n=3). b. Michaelis-Menten saturation curve of the basal enzymatic activity of recombinant ENO1wt, ENO1down and ENO1up in vitro in the absence of RNA using a non-linear curve fitting with least squares regression (n=3). c. Vmax and Km measurements for ENO1wt, ENO1down and ENO1up as determined from the Michaelis-Menten saturation curve in c. The asymmetrical confidence interval (CI) is given (n=3). d. Three distinct sets of target and control RNAs (5 μM) were nucleofected into mESCs. Upon nucleofection of the control or target RNAs, the lactate accumulation in the medium was measured after 30, 60 and 90 minutes and used to estimate the accumulation rate by calculating the slope. The R2 value was used as a quality control. The standard deviation is given and the two-tailed Student's t-test is used to detect statistically significant differences (n=3).

FIG. 9 shows riboregulation of ENO1 in HeLa cells. a. Relative enzymatic activity of ENO1wt, ENO1up, ENO1down and ENO1 as determined with recombinant proteins in vitro (n=3). b. Increasing concentrations of target and control PABPC1 were nucleofected into HeLa cells. Upon nucleofection of the control or target RNAs, the lactate accumulation in the medium was measured after 30, 60 and 90 minutes and used to estimate the accumulation rate (n=3). The standard deviation is given and the statistically significant differences were detected using two-way ANOVA and Sidak-corrected for multiple comparison testing. c. Same experimental setup as in b for FTH1 control and target RNA.

FIG. 10 shows analysis of ENO1 and mutant variants in mESCs. a. Lactate accumulation rate of pluripotent mESCs after LIF withdrawal for a period of seven days (n=3). The standard deviation is given and the two-tailed Student's t-test is used to detect statistically significant differences. b. Oxygen consumption rate of cells was measured with the Oxytherm System (n=3). The standard deviation is given and the two-tailed Student's t-test is used to detect statistically significant differences. c. Quantification of replicates of ENO1 PNK assays after UV-crosslinking for pluripotent mESCs and mouse cells after 7 days of LIF withdrawal (n=3). The standard deviation is given and the two-tailed Student's t-test is used to detect statistically significant differences. d. Immunoblot of ENO1, FLAG and HuR (loading control) for three clones for ENO1 knockout and constitutive transgenic expression of Flag-HA-tagged ENO1wt, ENO1up and ENO1down from the Rosa26 locus. e. The schematic shows that RNA riboregulates the enzymatic activity of ENO1, thereby inhibiting glycolysis and affecting mESC differentiation.

FIG. 11 shows that riboregulation of ENO1 affects mESC differentiation. a. Cells expressing Eomes-mCherry or Brachyury-BFP after seven days of LIF withdrawal were sorted for the respective fluorescent marker proteins, cultured for an additional five days and lactate accumulation in the culture medium was quantified (n=3). The standard deviation is given and the statistically significant differences were detected using the two-tailed Student's t-test. b. Cellular RNA-protein interactions were cross-linked with UV and a PNK assay was performed for cell populations described in a. A representative experiment is shown and was performed for a total of three times. Non-crosslinked (No CL), unsorted (us.) mESC were used as specificity controls. Immunoblots of ENO1 and actin are shown as controls. c. Cell lines were established for ENO1 knockout and constitutive transgenic expression of Flag-HA-tagged ENO1wt, ENO1up and ENO1down from the Rosa26 locus. RNA binding was measured for the three ENO1 variants by Flag immunoprecipitation and PNK assay after UV cross-linking in pluripotent mESCs. Three independent clones were used. The standard deviation is given and the statistically significant differences were detected using the two-tailed Student's t-test when comparing to ENO1wt. d. Measurement of the lactate accumulation in the culture medium for three independent clones of ENO1wt, ENO1up and ENO1down in pluripotent mESCs. The standard deviation is given and the statistically significant differences were detected using the two-tailed Student's t-test when comparing to ENO1wt. e.-g. Differentiation of mESCs and qPCRs of lineage marker for ENO1wt (e.), ENO1up (f.) and ENO1down (g.) after seven days of LIF withdrawal for three independent clones (from c.) in three experiments. The standard deviation is given and the statistically significant differences were detected using two-way ANOVA with Tukey-corrected multiple comparison testing when comparing to ENO1wt.

FIG. 12. Riboregulation of ENO1 affects mESC differentiation. A.-B. Cell lines were established for a transient ENO1 depletion using the auxin-inducible degron system. Four days after withdrawing LIF from the mESC medium, DMSO (A) or auxin (B) was added for 48 hours. The cells were differentiated for a total of seven days and RT-qPCRs were performed for differentiation and lineage marker. This experiment was performed with two independent clones in three biological replicates. The standard deviation is given, and the statistically significant differences were detected using two-way ANOVA with Sidak-corrected multiple comparison testing when comparing to the DMSO-treated samples. C. Immunoblot for auxin-induced degradation of ENO1 in two independent clones after 48 hours of treatment with auxin. Actin and Tubulin were stained as loading controls. D. Expression level differences of differentiation and lineage-relevant markers after treating wild-type mESCs with auxin or DMSO for seven days after withdrawing LIF. Statistically significant differences were detected using two-way ANOVA and Sidak-correction for multiple comparison testing (n=3).

FIG. 13 shows an analysis of ENO1 ubiquitination. a. TRIM21 (E3 ligase) co-immunoprecipitates (co-IP) with ENO1. b. Immunoprecipitation (IP) of ubiquitin shows enhanced ubiquitination of ENO1wt compared to ENO1up. The data supports that riboregulation of ENO1 is (at least partially) controlled by ubiquitination of ENO1.

FIG. 14 shows that acetylation activates ENO1's RNA binding. A. PNK assay of ENO1 in HeLa cells after a 16-hour treatment with the histone deacetylation (HDAC) inhibitor, sodium butyrate, with concentrations ranging from 2.5-10 mM. Western blotting was performed for ENO1 and using an antibody for acetylated lysine (acetyl-K). B. Western blot for SIRT2 and ENO1 after siRNA-mediated knockdown of SIRT2 in HeLa cells. C. Immunoprecipitation of ENO1 from siControl or siSIRT2-treated HeLa cells. Western blotting was performed for ENO1 and acetylated lysine (acetyl-K). D. Representative PNK assays for FLAG-tagged ENO1wt, ENO1up and ENO1down in HeLa cells after siRNA-mediated knockdown of SIRT2. Western blotting was performed for the FLAG-tagged ENO1 variants, SIRT2 to assess the knock-down efficiency, and Nucleolin as a loading control. E. Quantification of three biological replicates of the experimental setup in D. RNA binding as detected by the autoradiograph was normalized to the FLAG immunoprecipitation efficiency. Statistically significant differences were detected using two-way ANOVA and Sidak-correction for multiple comparison testing (SD, n=3). F. In vitro deacetylation assay coupled with mass spectrometry using recombinant SIRT2 wildtype or the enzymatically dead mutant SIRT2 H187Y and in vitro acetylated ENO1 wildtype. Two amino acids on the same peptide were found to respond to SIRT2 deacetylation. G. PNK assay of ENO1 in HeLa cells after a 24-hour treatment with 0.1% DMSO or the SIRT2 inhibitor, SirReal2 at a 12.5 μM. Western blotting was performed for ENO1 and nucleolin. H. PNK assay of ENO1 in HeLa cells after a 24-hour treatment with 0.1% DMSO or the SIRT2 inhibitor, Thiomyristoyl at a 10 μM. Western blotting was performed for ENO1 and nucleolin. I. Representative PNK assay for FLAG-tagged ENO1wt, ENO1up, ENO1down and an acetylation-mimicking version of the ENO1up mutant (ENO1KtoQ) in HeLa cells. Western blotting was performed for the FLAG-tagged ENO1 variants and Nucleolin as a loading control. J. Quantification of three biological replicates of the experimental setup in H. RNA binding as detected by the autoradiograph was normalized to the FLAG immunoprecipitation efficiency. Statistically significant differences were detected using two-way ANOVA and Sidak-correction for multiple comparison testing (SD, n=3).

FIG. 15 shows time- and germ layer-dependent changes in ENO1's RNA binding and acetylation level during mESC differentiation. A. Formaldehyde (0.1%)-crosslinked RNA-immunoprecipitation of ENO1 or an isotype-matched IgG from pluripotent mESCs, cells differentiated for three, five and seven days (-LIF). RT-qPCR for five specific and two control mRNAs. The RNA enrichment is calculated relative to the mean of the IgG samples and normalized to the input. Statistically significant differences were determined using two-way ANOVA and Sidak-correction for multiple comparison testing. B. Input for ENO1 immunoprecipitation in FIG. 15C. C. ENO1 immunoprecipitation from pluripotent mESCs, cells differentiated for three, five and seven days (-LIF), followed by immunoblotting using an antibody detecting acetylated lysine (acetyl-K). The ratio of acetyl-K signal in comparison to ENO1 staining was calculated for three biological replicates. The ratio of acetyl-K over ENO1 steadily increases and is significantly elevated at day seven (unpaired Student's T-test, p-value=0.030), highlighted in blue, relative to the ratio in pluripotent cells. D. Input for ENO1 immunoprecipitation in cells expressing Eomes-mCherry or Brachyury-BFP after seven days of LIF withdrawal were sorted for the respective fluorescent marker proteins, cultured for an additional five days and lysates were prepared. E. ENO1 immunoprecipitation in cells from FIG. 15D and staining with an antibody detecting acetylated lysine (acetyl-K). The ratio of acetyl-K signal in comparison to ENO1 staining was calculated for three biological replicates.

The sequences according to SEQ ID NOs. 1 to 14 show:

Amino acid sequence of human ENO1
SEQ ID NO: 1
MSILKIHAREIFDSRGNPTVEVDLFTSKGLFRAAVPSGAS
TGIYEALELRDNDKTRYMGKGVSKAVEHINKTIAPALVSK
KLNVTEQEKIDKLMIEMDGTENKSKFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
sequence motif 1
SEQ ID NO: 2
TTTTTTBTTTTTT
sequence motif 2
SEQ ID NO: 3
CCCAGRC
Amino acid sequence of human Polyadenylate-
binding protein cytoplasmic 1 (PABPC1)
SEQ ID NO: 4
MNPSAPSYPMASLYVGDLHPDVTEAMLYEKFSPAGPILSI
RVCRDMITRRSLGYAYVNFQQPADAERALDTMNFDVIKGK
PVRIMWSQRDPSLRKSGVGNIFIKNLDKSIDNKALYDTFS
AFGNILSCKVVCDENGSKGYGFVHFETQEAAERAIEKMNG
MLLNDRKVFVGRFKSRKEREAELGARAKEFTNVYIKNFGE
DMDDERLKDLFGKFGPALSVKVMTDESGKSKGFGFVSFER
HEDAQKAVDEMNGKELNGKQIYVGRAQKKVERQTELKRKF
EQMKQDRITRYQGVNLYVKNLDDGIDDERLRKEFSPFGTI
TSAKVMMEGGRSKGFGFVCFSSPEEATKAVTEMNGRIVAT
KPLYVALAQRKEERQAHLTNQYMQRMASVRAVPNPVINPY
QPAPPSGYFMAAIPQTQNRAAYYPPSQIAQLRPSPRWTAQ
GARPHPFQNMPGAIRPAAPRPPFSTMRPASSQVPRVMSTQ
RVANTSTQTMGPRPAAAAAAATPAVRTVPQYKYAAGVRNP
QQHLNAQPQVTMQQPAVHVQGQEPLTASMLASAPPQEQKQ
MLGERLFPLIQAMHPTLAGKITGMLLEIDNSELLHMLESP
ESLRSKVDEAVAVLQAHQAKEAAQKAVNSATGVPTV
Amino acid sequence of human Ferritin
heavy chain 1 (FTH1) protein
SEQ ID NO: 5
MTTASTSQVRQNYHQDSEAAINRQINLELYASYVYLSMSY
YFDRDDVALKNFAKYFLHQSHEEREHAEKLMKLQNQRGGR
IFLQDIKKPDCDDWESGLNAMECALHLEKNVNQSLLELHK
LATDKNDPHLCDFIETHYLNEQVKAIKELGDHVTNLRKMG
APESGLAEYLFDKHTLGDSDNES
Amino acid sequence of human Protein
tyrosine phosphatase type IVA
(PTP4A1)
SEQ ID NO: 6
MARMNRPAPVEVTYKNMRFLITHNPTNATLNKFIEELKKY
GVTTIVRVCEATYDTTLVEKEGIHVLDWPFDDGAPPSNQI
VDDWLSLVKIKFREEPGCCIAVHCVAGLGRAPVLVALALI
EGGMKYEDAVQFIRQKRRGAFNSKQLLYLEKYRPKMRLRF
KDSNGHRNNCCIQ
Amino acid sequence of ENO1-K89A mutant
SEQ ID NO: 7
MSILKIHAREIFDSRGNPTVEVDLFTSKGLFRAAVPSGAS
TGIYEALELRDNDKTRYMGKGVSKAVEHINKTIAPALVSK
KLNVTEQEAIDKLMIEMDGTENKSKFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1-K92A mutant
SEQ ID NO: 8
MSILKIHAREIFDSRGNPTVEVDLFTSKGLFRAAVPSGAS
TGIYEALELRDNDKTRYMGKGVSKAVEHINKTIAPALVSK
KLNVTEQEKIDALMIEMDGTENKSKFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1-K105A mutant
SEQ ID NO: 9
MSILKIHAREIFDSRGNPTVEVDLFTSKGLFRAAVPSGAS
TGIYEALELRDNDKTRYMGKGVSKAVEHINKTIAPALVSK
KLNVTEQEKIDKLMIEMDGTENKSAFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1-K89A/K92A
mutant
SEQ ID NO: 10
MSILKIHAREIFDSRGNPTVEVDLFTSKGLFRAAVPSGAS
TGIYEALELRDNDKTRYMGKGVSKAVEHINKTIAPALVSK
KLNVTEQEAIDALMIEMDGTENKSKFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1-K89A/K105A
mutant
SEQ ID NO: 11
MSILKIHAREIFDSRGNPTVEVDLFTSKGLFRAAVPSGAS
TGIYEALELRDNDKTRYMGKGVSKAVEHINKTIAPALVSK
KLNVTEQEAIDKLMIEMDGTENKSAFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1-K92A/K105A
mutant
SEQ ID NO: 12
MSILKIHAREIFDSRGNPTVEVDLFTSKGLFRAAVPSGAST
GIYEALELRDNDKTRYMGKGVSKAVEHINKTIAPALVSKK
LNVTEQEKIDALMIEMDGTENKSAFGANAILGVSLAVCKA
GAVEKGVPLYRHIADLAGNSEVILPVPAFNVINGGSHAGN
KLAMQEFMILPVGAANFREAMRIGAEVYHNLKNVIKEKYG
KDATNVGDEGGFAPNILENKEGLELLKTAIGKAGYTDKVV
IGMDVAASEFFRSGKYDLDFKSPDDPSRYISPDQLADLYK
SFIKDYPVVSIEDPFDQDDWGAWQKFTASAGIQVVGDDLT
VTNPKRIAKAVNEKSCNCLLLKVNQIGSVTESLQACKLAQ
ANGWGVMVSHRSGETEDTFIADLVVGLCTGQIKTGAPCRS
ERLAKYNQLLRIEEELGSKAKFAGRNFRNPLAK
Amino acid sequence of ENO1-K89A/K92A/
K105A mutant
SEQ ID NO: 13
MSILKIHAREIFDSRGNPTVEVDLFTSKGLFRAAVPSGAS
TGIYEALELRDNDKTRYMGKGVSKAVEHINKTIAPALVSK
KLNVTEQEAIDALMIEMDGTENKSAFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLKVNQIGSVTESLQACKLA
QANGWGVMVSHRSGETEDTFIADLVVGLCTGQIKTGAPCR
SERLAKYNQLLRIEEELGSKAKFAGRNFRNPLAK
Amino acid sequence of ENO1- K343A mutant
SEQ ID NO: 14
MSILKIHAREIFDSRGNPTVEVDLFTSKGLFRAAVPSGAS
TGIYEALELRDNDKTRYMGKGVSKAVEHINKTIAPALVSK
KLNVTEQEKIDKLMIEMDGTENKSKFGANAILGVSLAVCK
AGAVEKGVPLYRHIADLAGNSEVILPVPAFNVINGGSHAG
NKLAMQEFMILPVGAANFREAMRIGAEVYHNLKNVIKEKY
GKDATNVGDEGGFAPNILENKEGLELLKTAIGKAGYTDKV
VIGMDVAASEFFRSGKYDLDFKSPDDPSRYISPDQLADLY
KSFIKDYPVVSIEDPFDQDDWGAWQKFTASAGIQVVGDDL
TVTNPKRIAKAVNEKSCNCLLLAVNQIGSVTESLQACKLA
QANGWGVMVSHRSGETEDTFIADLVVGLCTGQIKTGAPCR
SERLAKYNQLLRIEEELGSKAKFAGRNFRNPLAK

Mutated amino acids in SEQ ID NOs: 7 to 14 are highlighted. The mutated amino acids in SEQ ID NOs: 7 to 14 are Lysine to Alanine mutations, when compared to the sequence of SEQ ID NO: 1.

### EXAMPLES

Certain aspects and embodiments of the invention will now be illustrated by way of example and with reference to the description, figures and tables set out herein. Such examplesrefe of the methods, uses and other aspects of the present invention are representative only, and should not be taken to limit the scope of the present invention to only such representative examples.

The examples show:

### Example 1: Enolase 1 Binds to Specific Transcriptomic Sites

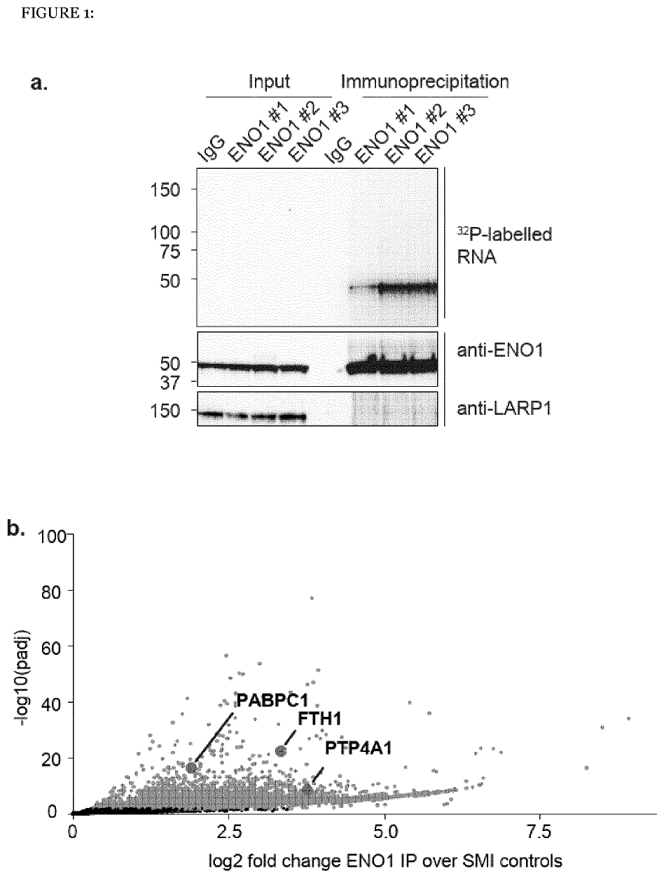

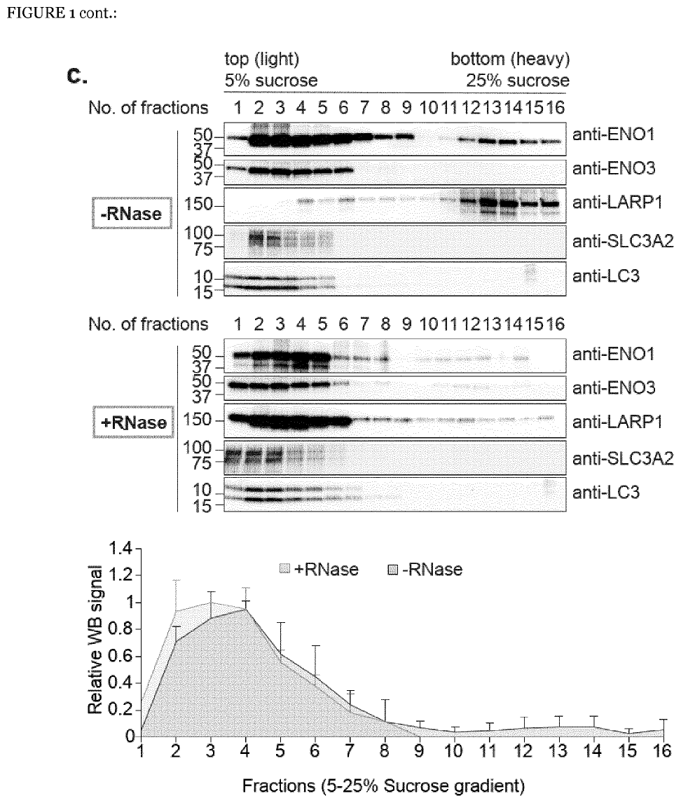

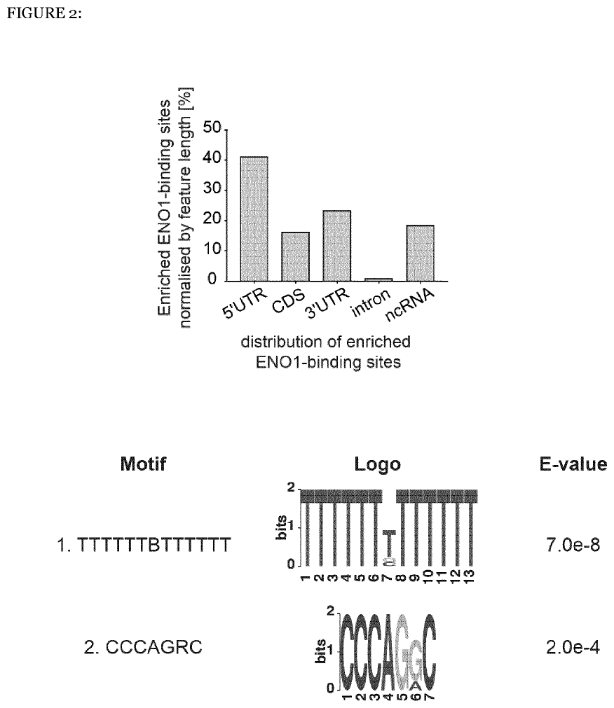

The inventors used a PNK assay [1] to confirm that human ENO1 binds RNA in HeLa cells (FIG. 1a). The inventors estimated that under basal conditions around 10% of HeLa cell ENO1 is sensitive to RNase treatment when exposing RNase-treated or untreated lysates to sucrose density gradient centrifugation (FIG. 1c). The inventors also determined the RNA-binding sites of ENO1 in the transcriptome by applying an enhanced crosslinking and immunoprecipitation (eCLIP) protocol [2] CLIP of ENO1, which enabled to identify RNA-binding sites on a transcriptome-wide scale, and revealed RNA-binding sites at nucleotide resolution. The inventors attained that ENO1 interacts with a wide range of RNAs in HeLa cells with a preference towards the 5′untranslated region (5′UTR) of mRNAs (FIG. 2). Based on the exact crosslinking sites, the inventors identified approximately two thousand direct ENO1-binding sites across the transcriptome (FIG. 1b) that do not display striking linear sequence motif recognition. The top scoring two sequence motifs (FIG. 2: TTTTTTBTTTTTT, and CCCAGRC) jointly account for only ˜22% of all ENO1 binding sites.

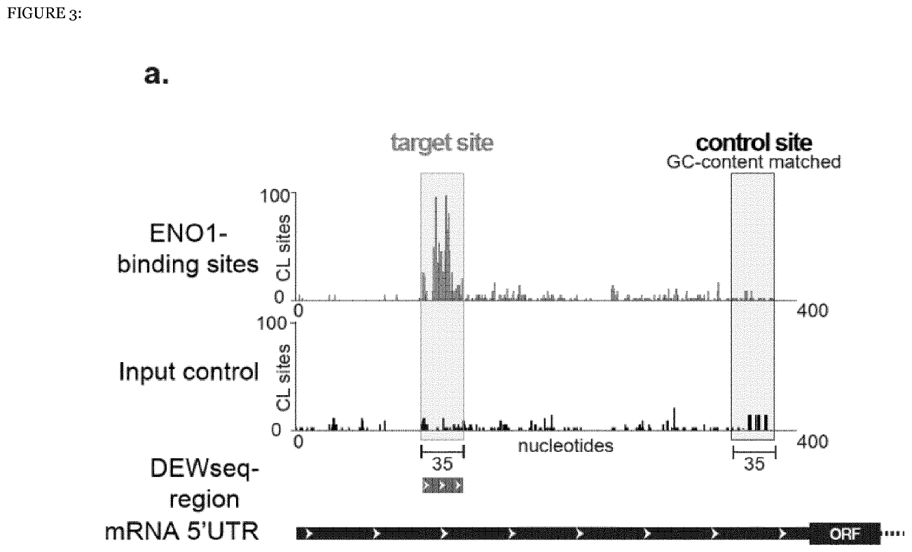

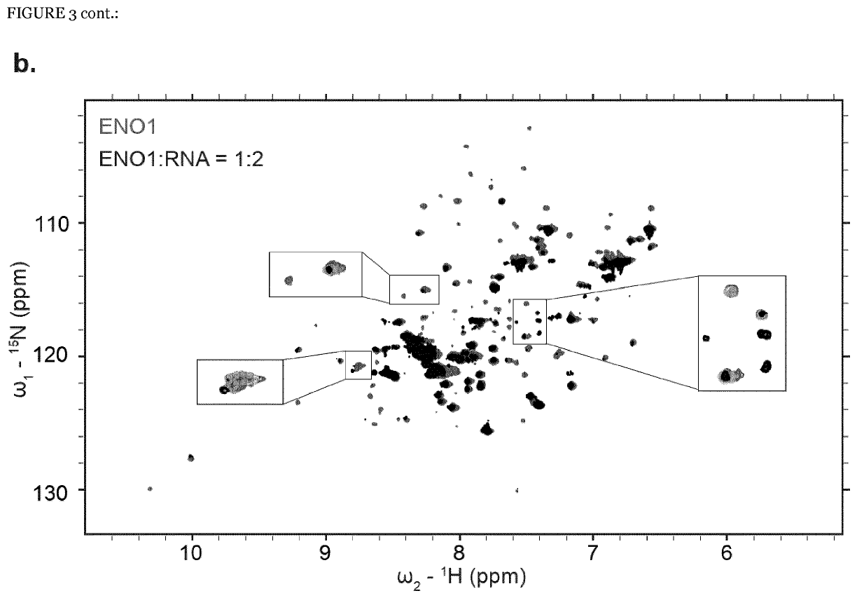

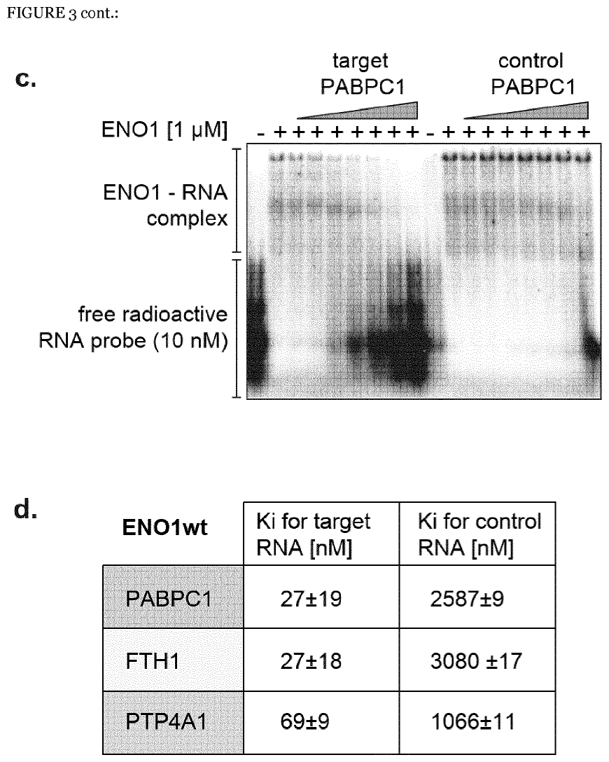

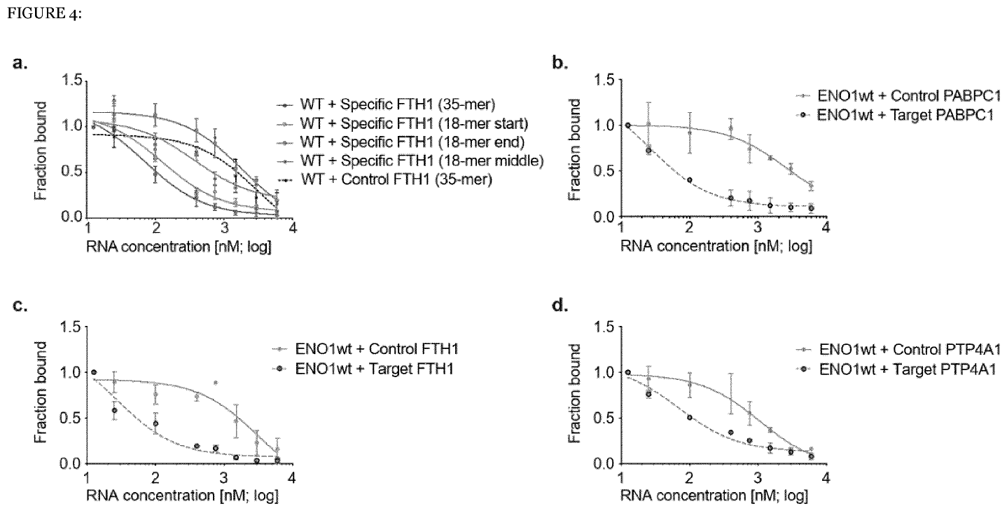

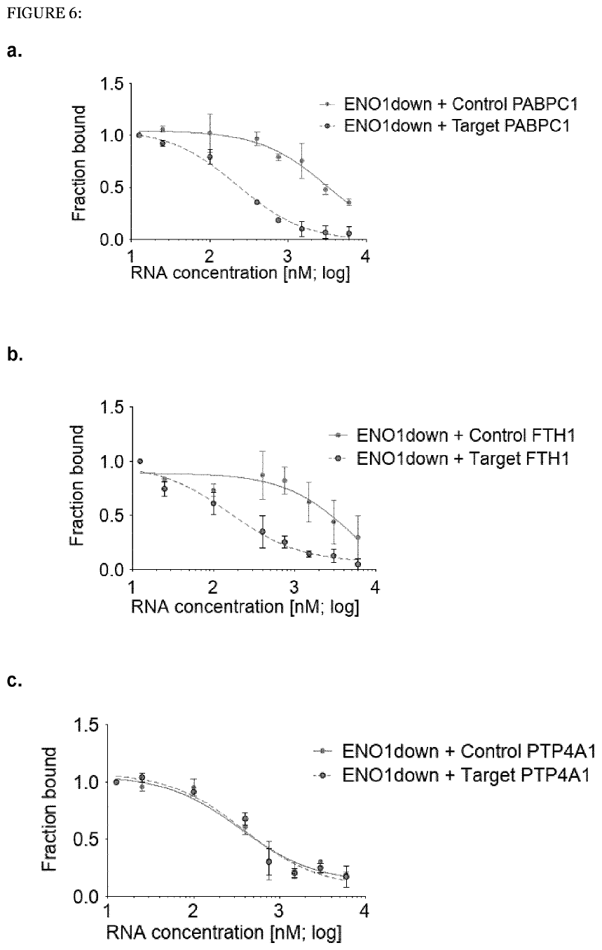

For validation experiments, the inventors synthesized RNAs of 35 nucleotides in length that either correspond to ENO1's binding site or a GC-matched control, derived from a region of the same mRNA downstream from its binding site (schematic in FIG. 3a). The inventors tested an exemplary target and control RNA, derived from the PABPC1 5′UTR in a competition electromobility shift assay (EMSA) using recombinant human ENO1 (FIGS. 3c and d; Ki_{target}:27±19 nM; Kicontrol: 2587±9 nM, FIG. 6). Similar results were obtained for two additional target and control pairs derived from the PTP4A1 and FTH1 mRNAs, respectively (FIG. 3d, FIG. 6a and b). Using NMR, the inventors observed RNA-induced chemical shift perturbations and line broadening of ENO1 resonances in ¹H, ¹⁵N-HSQC spectra, confirming direct RNA binding in vitro (shortened FTH1 target RNA (18-mer), FIG. 3b). Taken together, the inventors showed that ENO1 specifically binds RNA targets, in particular, ENO1 binds RNA at numerous transcriptomic sites in human cells with two orders of magnitude difference between specific and non-specific interactions.

### Example 2: Development of ENO1 Mutants Characterized by Enhanced and/or Decreased RNA Binding to ENO1

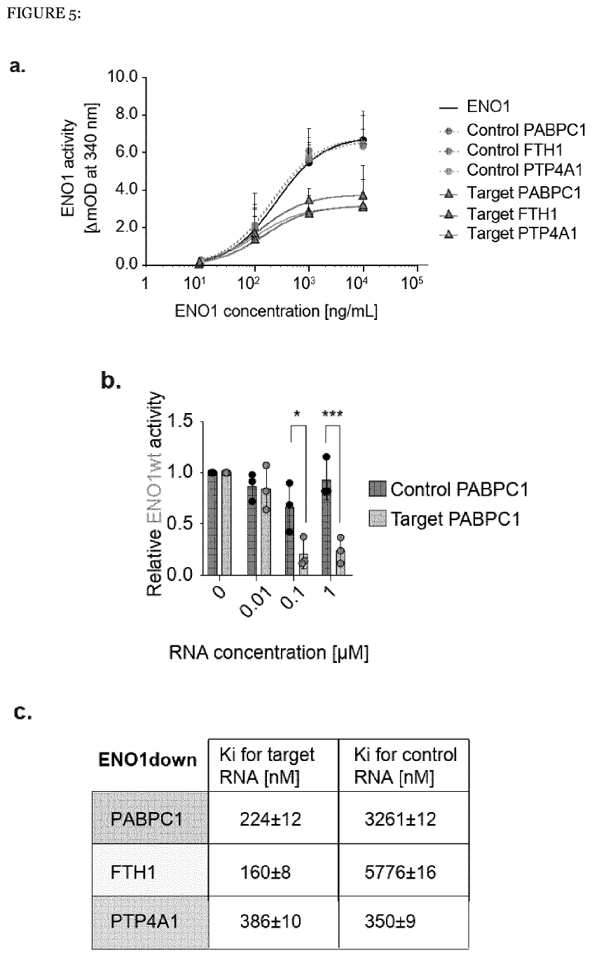

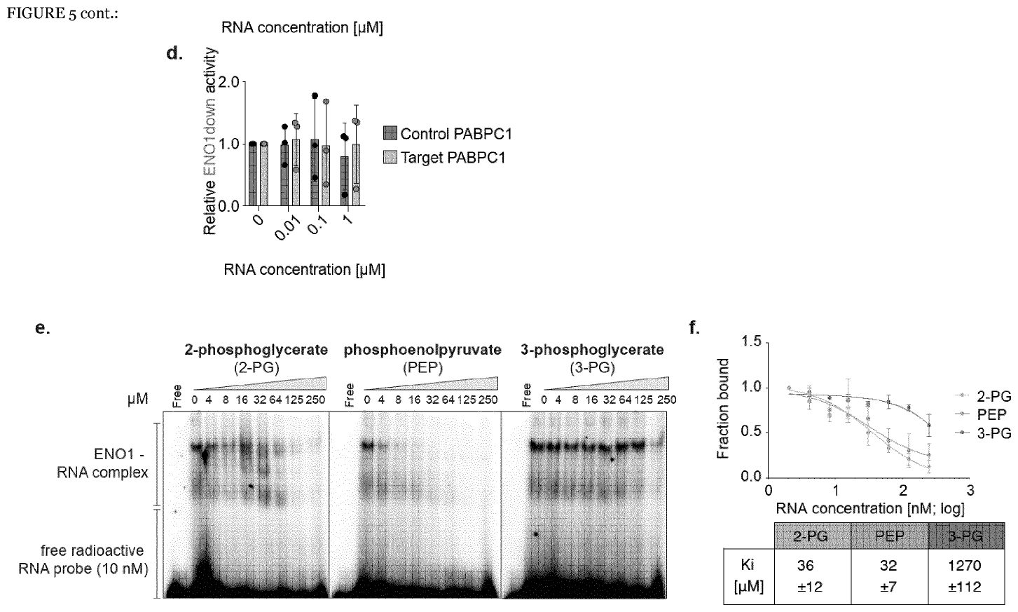

Instructed by RBDmap data, the inventors further generated an ENO1 mutant (K343A; ENO1down) with ˜5-10-fold decreased RNA binding (FIGS. 5c and d; Kitarget: 224±12 nM; Kicontrol: 3261±12 nM, FIG. 6) compared to ENO1wt as measured by competitive EMSA (FIGS. 5c and d, FIG. 5). Importantly, ENO1down displays no discernible alteration in its enzymatic activity with any of the RNAs tested (FIG. 5d, FIG. 6). Thus, both control RNAs and the ENO1 down mutant corroborate that RNA specifically riboregulates ENO1's activity in vitro.

Next, the inventors tested whether ENO1's enzymatic substrate binding and RNA binding are competitive. For this reason, the inventors performed EMSA experiments utilizing 2-PG and PEP as competitors. While both substrates compete with RNA for ENO1's binding, the specificity control 3-phosphoglycerate (3-PG), the immediate precursor of 2-PG with an identical molecular mass fails to compete (FIGS. 5e and f).Thus, the inventors observe specific competition between substrates and RNA targets for binding to ENO1. The inventors' data demonstrate that ENO1's enzymatic activity is reduced when incubated with specific RNAs, supporting a role of RNA as a regulator of ENO1.

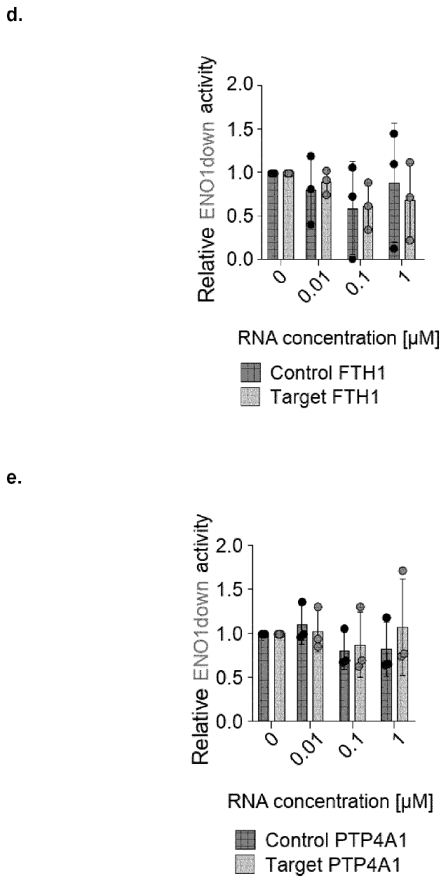

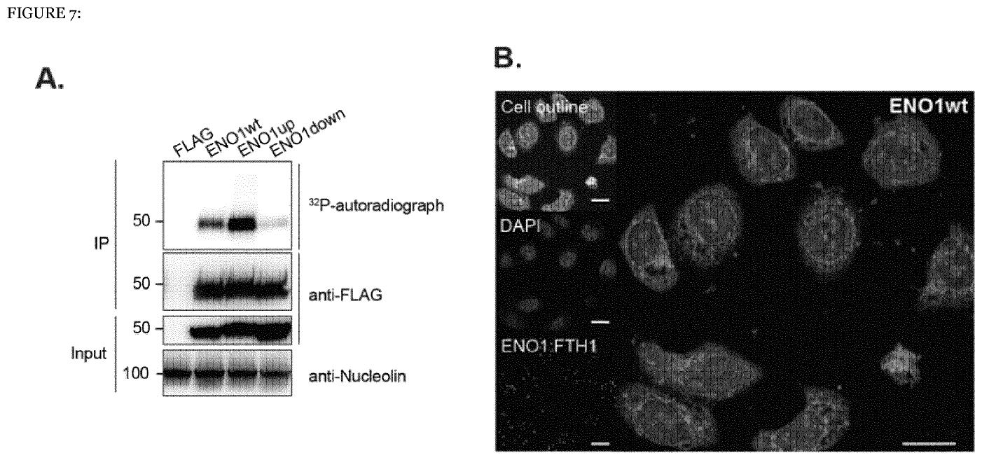

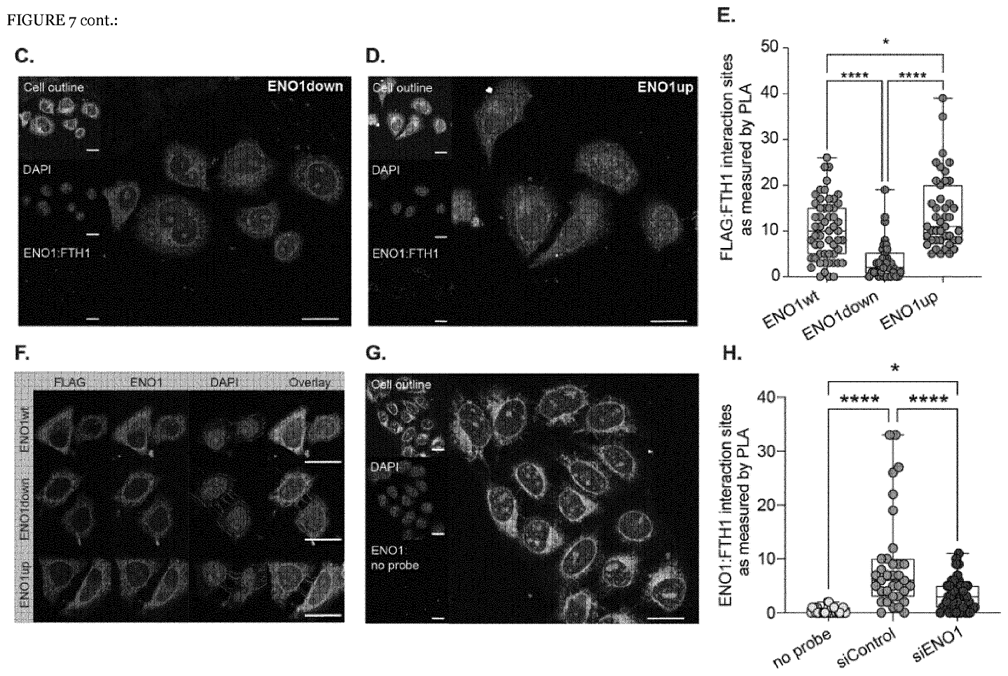

In addition to ENO1down, the inventors generated an ENO1up mutant. The design of ENO1up was also guided by RBDmap data and entails the change of lysine residues (K89A/K92A/K105A) along the inferred interaction region of RNA with the enzyme. After knocking down endogenous HeLa cell ENO1 and rescue with the respective Flag-tagged ENO1 variant, ENO1up displays increased RNA binding compared to ENO1wt, as measured by PNK assays (FIG. 7a). In line with the in vitro data, ENO1down displays substantially decreased RNA binding in HeLa cells relative to ENO1wt (FIG. 7a).

The inventors independently confirmed the differential RNA binding of the ENO1up and ENO1down mutants using an immunofluorescence-based, UV crosslinking-independent RNA proximity ligation assay (PLA), as disclosed in [3] (Zhang et al., 2016) that enables the in situ detection of endogenous or tagged proteins with their RNA targets. ENO1's association with the FTH1 mRNA ligand was validated by the combination of an antisense probe hybridizing close to ENO1's FTH1 mRNA-interaction site and an antibody specifically recognizing the Flag-tagged ENO1 variants. Using this orthogonal assay, the inventors validated the differential RNA binding of ENO1wt (FIG. 7B, E), ENO1down (FIG. 7C, E) and ENO1up (FIG. 7D, E) in HeLa cells, ensuring that the expression levels and localization of the ENO1 variants were comparable (FIG. 7F) and that the PLA signal is specific (FIG. 7G and H).

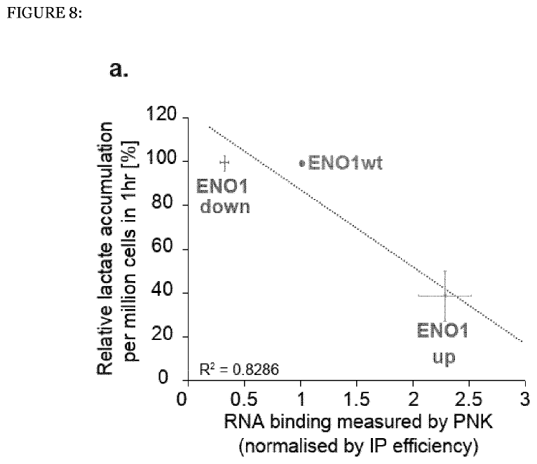

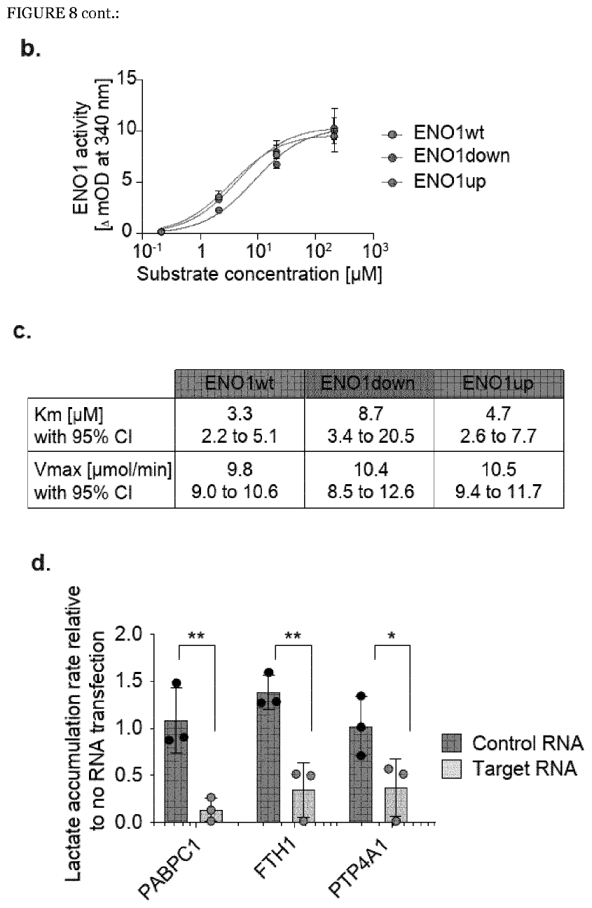

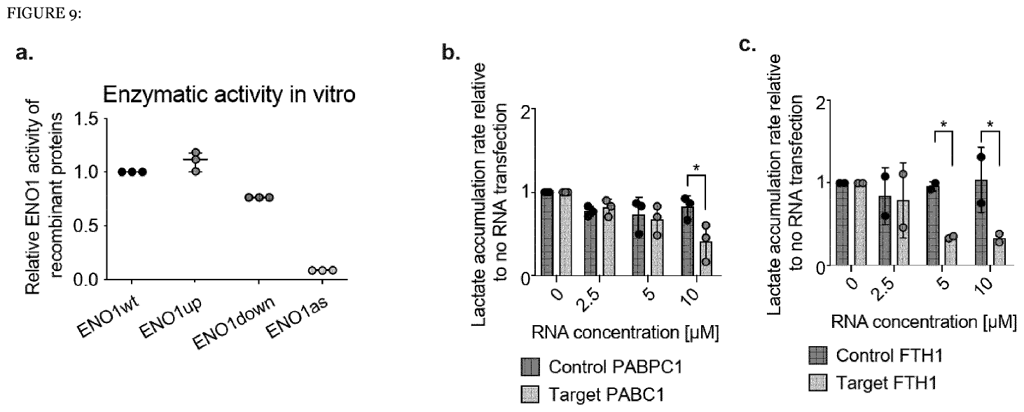

When the inventors tested the ENO1down and ENO1up mutants for their ability to rescue glycolysis (lactate accumulation in the medium) in HeLa cells after knock-down of the endogenous ENO1, ENO1wt rescued lactate production (FIG. 8a). Of note, the RNA binding-deficient mutant ENO1down is as active as the wild-type protein (FIG. 8a), showing that the K343A mutation does not incapacitate the enzyme. In contrast, ENO1up fails to rescue the knock-down-induced inhibition of lactate accumulation (FIG. 8a), although it is fully active when tested in the absence of RNA in vitro (FIGS. 8b and c, FIG. 9a). The activity measurements were controlled with a mutant lacking enzymatic activity (ENO1as, E295A/D320A/K394A, FIG. 9a). These results are consistent with the notion that ENO1's RNA binding interferes with its enzymatic activity in cells.

The inventors complemented these experiments by nucleofection of target and control synthetic 35-mer RNAs into HeLa cells. The results unambiguously confirm specific riboregulation of lactate production by the ENO1 target RNAs tested (FIGS. 9b and c). Accordingly, the inventors showed that RNA ligands inhibit ENO1's enzymatic activity in vitro, and ENO1's enzymatic substrates specifically compete with its RNA binding. Increasing the concentration of RNA ligands in cultured cells inhibits glycolysis.

### Example 3: Riboregulation of ENO1 Affects Mouse Embryonic Stem Cell Differentiation

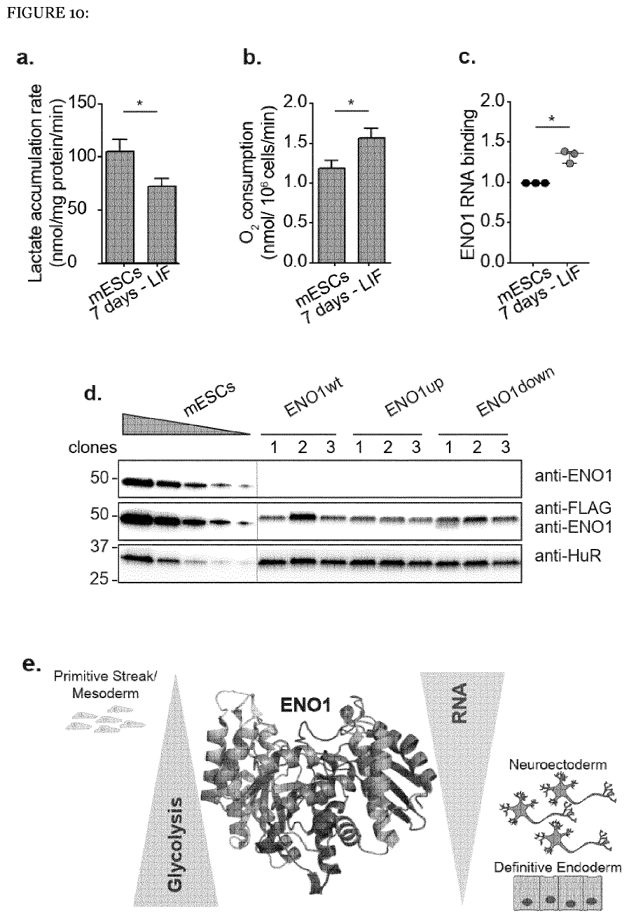

To explore physiological functions of the ENO1-RNA interaction, the inventors chose mouse embryonic stem cells (mESCs). Like many cancer cells, mESCs utilizes glucose as a major energy source in the undifferentiated state. Removal of the leukaemia inhibitory factor (LIF) from the culture medium induces differentiation, accompanied by a decrease in glycolysis and increased respiration (FIGS. 10a and b). Interestingly, the decrease in glycolysis correlates with increased ENO1 RNA binding after LIF withdrawal (FIG. 10c).

To directly test the effect of RNA on ENO1 in mESCs, the inventors nucleofected control or target RNAs and measured lactate accumulation in the medium. Confirming the results from HeLa cells (FIGS. 9b and c), all three specific ENO1-binding RNAs significantly reduce lactate accumulation, in stark contrast to the control RNAs (FIG. 8d).

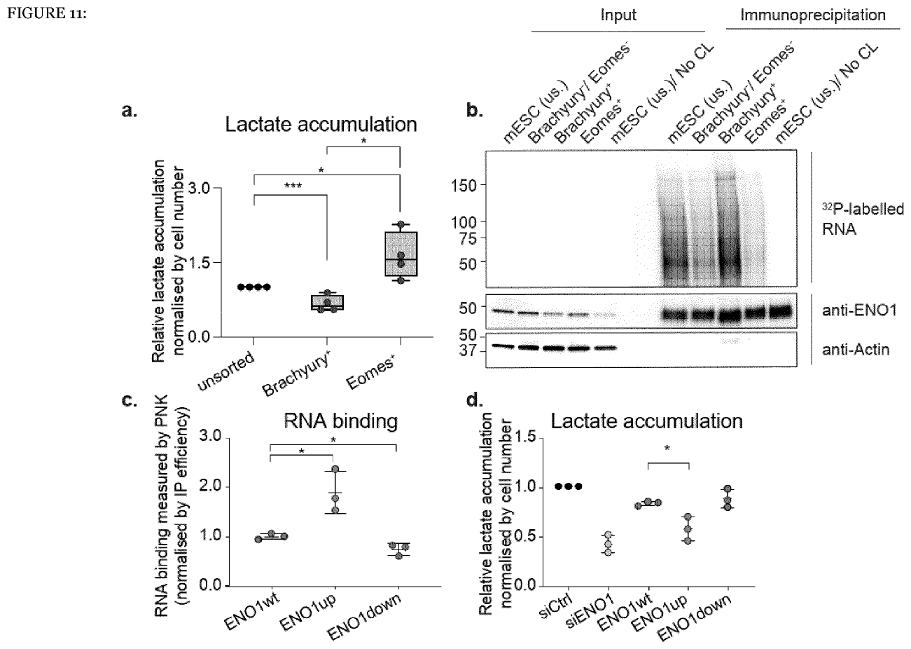

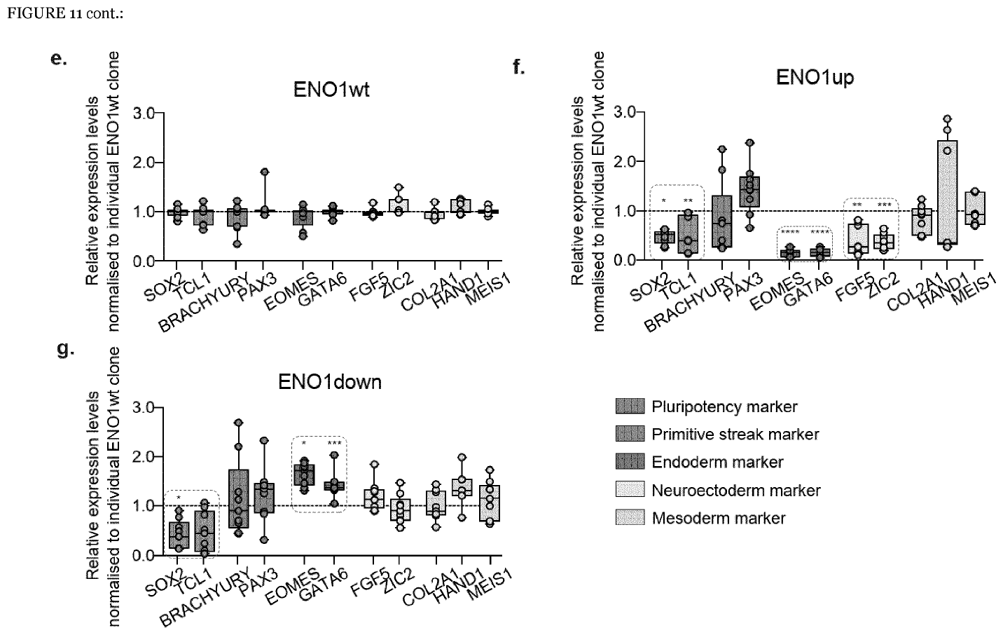

Unfortunately, the RNA nucleofection protocol is incompatible with meaningful mESC differentiation analyses. For this reason, the inventors used unperturbed mESCs, withdrew LIF for a period of seven days, and sorted cells that were positive for the expression of Brachyury (BFP-positive), which is primarily found in cells differentiating towards the primitive streak, or Eomes (mCherry-positive), which is predominantly expressed in the definitive endoderm. The inventors detected that lactate accumulation in the medium of Eomes+cells significantly exceeds that of Brachyury+cells (FIG. 11a), suggesting that the differentiation to the definitive endoderm may require sustained glycolysis in comparison to the primitive streak. Of note, ENO1 binding to RNA correlates inversely (compare FIGS. 11a and b).

To examine whether this correlation reflects a causal requirement for riboregulation of ENO1 during ESC differentiation, the inventors knocked out endogenous ENO1 and introduced murine versions of the ENO1 variants, characterized above, into the Rosa26 locus by CRISPR/Cas9 genome editing. The different heterologous forms of ENO1 are expressed at similar levels and below the expression level of endogenous ENO1 (FIG. 10d). As one would expect, lactate accumulation in the medium of ENO1wt cells is somewhat less than seen in control cells (FIG. 11d). As previously observed in HeLa cells, ENO1up displays increased RNA binding and mediates decreased lactate production; likewise, ENO1down shows a decrease in RNA binding compared to ENO1wt (FIGS. 11c and d).

Independent clones of these cell lines were subjected to LIF withdrawal and analysed for differentiation to the different germ layers. Engineered mESCs expressing ENO1wt differentiated normally into the distinct germ layers, as assessed by qPCR analysis of the respective expression of marker genes (FIG. 11e). By contrast, ENO1up-expressing cells fail profoundly in their differentiation to definitive endoderm and neuroectoderm (FIG. 11f), while the expression of primitive streak and mesodermal markers was quite variable and statistically not significantly affected. The inventors also noticed that ENO1down cells, where ENO1's activity escapes riboregulation, show increased differentiation towards the definitive endoderm (FIG. 11g).

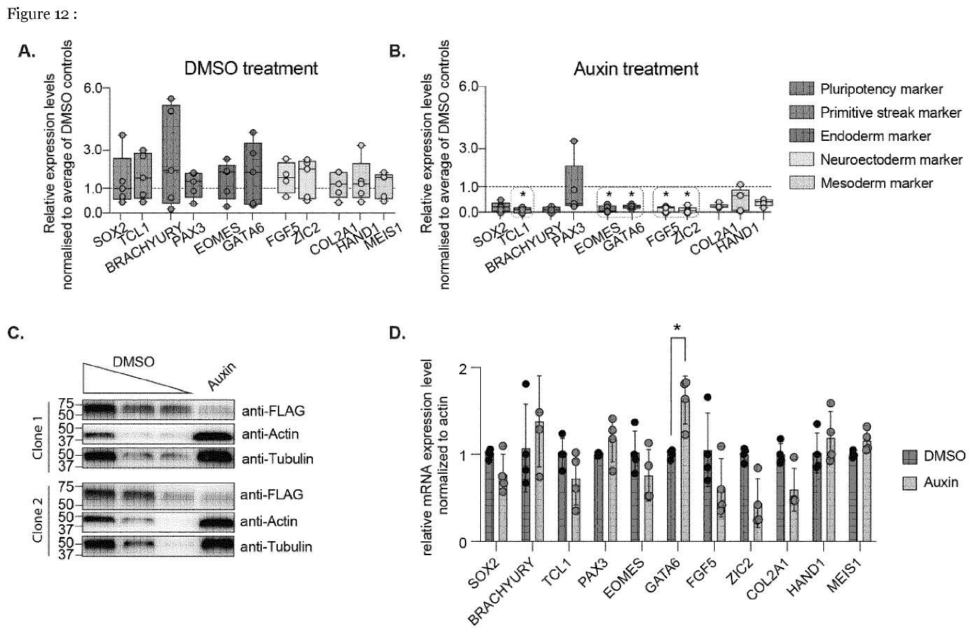

To corroborate that the phenotypic changes of ENO1up-expressing cells is a consequence of diminished ENO1 activity, the inventors fused an auxin-inducible degron tag to the C-terminus of endogenous ENO1 of both alleles in mESCs carrying the OsTir1 receptor in the TIGRE locus. The inventors then triggered ENO1 degradation by the addition of auxin for 48 hours at the previously determined critical point of differentiation of four days, where the inventors detected an increase in ENO1's RNA association. When depleting ENO1 from differentiating mESCs at this point, the inventors observe specific, defective differentiation towards neuroectoderm and definitive endoderm, phenocopying cells expressing ENO1up (FIGS. 12A and B).

Taken together, the inventors' results disclose a physiological ENO1 riboregulation in the course of stem cell differentiation, such as mESC differentiation, and its requirement for the formation of specific germ layers, especially for the formation of the endodermal germ layer. Importantly, pluripotent stem cells expressing an ENO1 mutant that is hyper-inhibited by RNA are severely impaired in their glycolytic capacity and in endodermal differentiation, whereas cells with an RNA binding-deficient ENO1 mutant display disproportionately high endodermal marker expression. As such, these results represent a novel form of regulated cell differentiation.

### Example 4: Analysis of ENO1 Ubiquitination

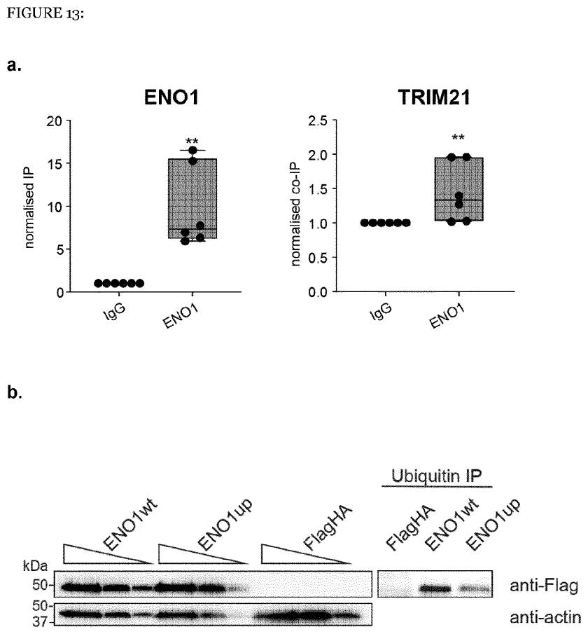

Interestingly, TRIM21 (E3 ligase) co-immunoprecipitates with ENO1 (FIG. 13a). Based on Mass Spectrometry data, the inventors discovered that K89 and K92 of ENO1 are ubiquitinated, which overlap with the amino acids mutated for ENO1up ENO1-K89A/K92A/K105A mutant). Also, immunoprecipitation (IP) of ubiquitin revealed enhanced ubiquitination of ENO1wt compared to ENO1up (FIG. 13b). Therefore, the inventors analyzed whether riboregulation of ENO1 is (at least partially) controlled by at least one posttranslational modification of ENO1, such as ubiquitination. According to this data, the transcriptome as a whole bearing thousands of relevant ENO1-binding regions would serve a very specific regulatory function, and influencing, e.g., ubiquitination can modulate RNA binding to ENO1, and, thereby, allow therapeutic intervention.

### Example 5: Acetylation Augments ENO1's RNA Binding

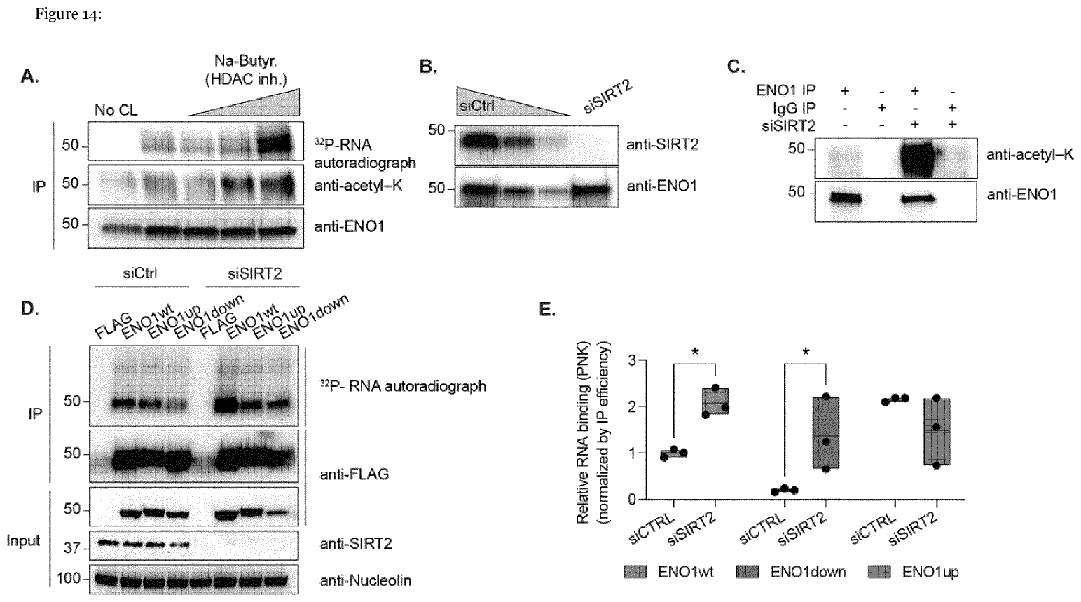

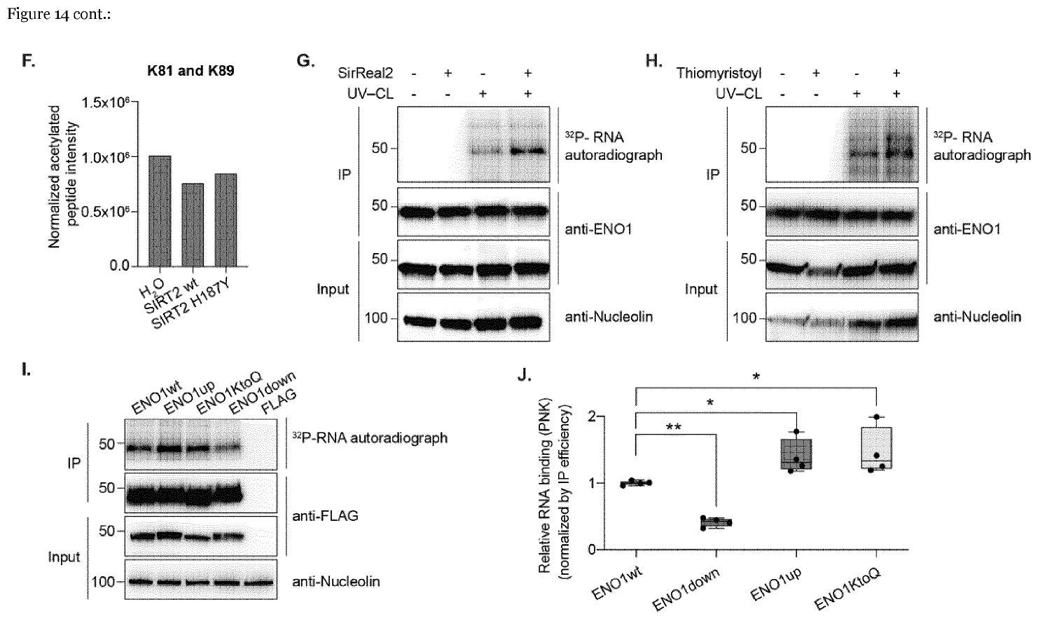

The inventors questioned what may explain the difference between the enhanced RNA binding of the ENO1up mutant in cellulo and the normal RNA binding of the recombinant protein in vitro. Considering that the ENO1up mutant represents a change of three lysine residues to alanine, the inventors hypothesised that a post-translational lysine modification such as ubiquitination or acetylation in cellulo could activate ENO1's RNA binding. Interestingly, treatment of HeLa cells with sodium butyrate, an inhibitor of protein deacetylases, profoundly induced ENO1's acetylation and RNA binding (FIG. 14A).

SIRT2 had previously been implicated in the deacetylation of ENO1. Thus, the inventors knocked down SIRT2′s expression with siRNAs and assessed the consequences on the RNA-binding of wild-type ENO1 and the ENO1 mutants that the inventors had generated. RNA binding of ENO1wt and ENO1down increased when knocking down ENO1's putative deacetylase, while ENO1up remained unaffected, suggesting that the ENO1up mutation mimics acetylated ENO1 (FIGS. 14B and C). To test this possibility more directly, the inventors mutated the lysines that are changed to alanine in ENO1up (K89, K92 and K105) to the more conventionally used acetylation-mimic, glutamine (ENO1KtoQ). In support of the above results and interpretation, ENO1KtoQ shows the same enhancement of RNA binding as ENO1up compared to ENO1wt (FIGS. 14D and E). While these lysines might not be in direct contact with RNA, acetylation at these positions may cause a conformational change opening the protein for RNA binding.

### Example 6: ENO1's RNA Association Increases During Differentiation

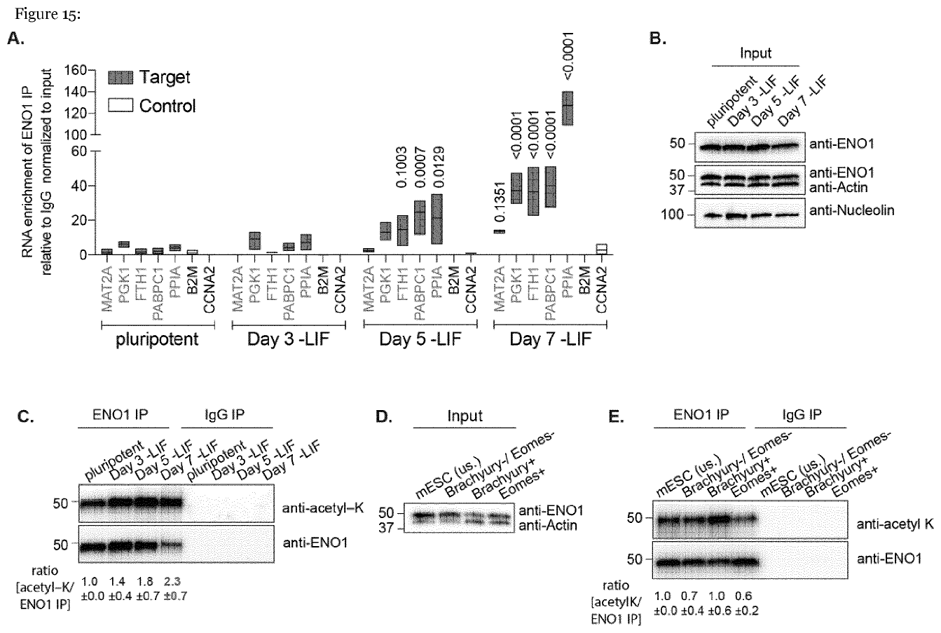

To temporally resolve the increase in RNA binding over the course of mESC differentiation, the inventors performed RIP-RT-qPCR experiments for the ligand and control mRNAs previously validated in HeLa cells. The inventors detected only minimal changes in ENO1's RNA association during the first three days, a modest increase in its binding to some of the ligands after five days (FIG. 15A, FTH1, PABPC1 and PPIA), and a pronounced, significant enrichment for four of the five ligands after seven days without LIF (FIG. 15A). This rise in RNA binding during mESC differentiation is accompanied and may be caused by an increase in ENO1's acetylation (FIG. 15C).

### REFERENCES

The references are:
[1] Richardson, C. C. Phosphorylation of nucleic acid by an enzyme from T4 bacteriophage-infected Escherichia coli. Proc. Natl. Acad. Sci. 54, 158-165 (1965).
[2] Van Nostrand, E. L. et al. Robust transcriptome-wide discovery of RNA-binding protein binding sites with enhanced CLIP (eCLIP). Nat. Methods 13, 508-514 (2016).
[3] Zhang, W., Xie, M., Shu, M.-D., Steitz, J. A., and DiMaio, D. (2016). A proximity-dependent assay for specific RNA-protein interactions in intact cells. RNA 22, 1785-1792.

1. A method for identifying and/or characterizing a compound suitable for the prevention and/or treatment of a disease, the method comprising the steps of:
(a) Providing at least one enzyme of the glycolytic pathway, at least one nucleic acid, and a candidate compound;
(b) Bringing into contact the at least one enzyme of the glycolytic pathway, the at least one nucleic acid and the candidate compound;
(c) Detecting and/or quantifying a binding between the at least one enzyme of the glycolytic pathway and the at least one nucleic acid;
wherein a differential level of the binding between the at least one enzyme of the glycolytic pathway and the at least one nucleic acid contacted with the candidate compound compared to the binding between the at least one enzyme of the glycolytic pathway and the at least one nucleic acid not contacted with the candidate compound indicates the candidate compound as suitable for the prevention and/or treatment of the disease.

2. The method according to claim 1, wherein steps (b) and (c) are performed in a cell-free system, or in a cell, such as in a biological assay cell or in a cell derived from a biological sample, such as a tissue sample or a body liquid sample of a subject, for example a blood sample.

3. The method according to claim 1 or 2, wherein the at least one nucleic acid is a functional or non-functional RNA polynucleotide molecule or a functional or non-functional DNA polynucleotide molecule, such as a single-stranded or doubled-stranded RNA polynucleotide molecule or DNA polynucleotide molecule, or a fragment or derivative thereof, for example an mRNA molecule, an RNA mimic, an RNA precursor, an RNA analogue, an RNA antisense molecule, an inhibitory RNA molecule, a ribozyme, an RNA antisense expression molecule, an RNA interference (RNAi) molecule, an siRNA molecule, an esiRNA molecule, an shRNA molecule, a miRNA molecule, a DNA mimic, a DNA precursor, a DNA analogue, an antisense DNA, a DNA aptamer, a decoy molecule, a GapmeR, a PNA (peptide nucleic acid) molecule, an LNA molecule (locked nucleic acid), a genetic construct for targeted gene editing, such as a CRISPR/Cas9 construct, a guide nucleic acid (gRNA or gDNA), and/or a tracrRNA, optionally wherein the at least one nucleic acid comprises at least one modification, for example a chemical modification selected from a modified internucleoside linkage, a modified nucleobase, or a modified sugar moiety, such as a 2′-O-alkyl modification, for example a 2′-O-methoxy-ethyl (MOE) or 2′-O-Methyl (OMe) modification, an ethylene-bridged nucleic acid (ENA), a 2′-fluoro (2′-F) nucleic acid, such as 2′-fluoro N3-P5′-phosphoramidite, a 1′,5′-anhydrohexitol nucleic acid (HNA), or a locked nucleic acid (LNA).

4. The method according to any one of claims 1 to 3, wherein the candidate compound is selected from a small molecular compound (“small molecule”), a polypeptide, a peptide, a glycoprotein, a peptidomimetic, an antigen binding construct (for example, an antibody, antibody-like molecule or other antigen binding derivative, or an antigen binding fragment thereof), a nucleic acid, such as a DNA or RNA, for example an antisense or inhibitory DNA or RNA, a ribozyme, an RNA or DNA aptamer, RNAi, siRNA, shRNA and the like, including variants or derivatives thereof, such as a peptide nucleic acid (PNA), a genetic construct for targeted gene editing, such as a CRISPR/Cas9 construct, a guide nucleic acid (gRNA or gDNA), and/or a tracrRNA.

5. The method according to any one of claims 1 to 4, wherein the detecting and/or quantifying in step (c) involves at least one of:
(i) UV cross-linking, immunoprecipitation and radioactive labelling of co-purified RNA (PNK assay);
(ii) Enhanced Crosslinking and Immunoprecipitation (eCLIP);
(iii) Photoactivatable Ribonucleoside-Enhanced Crosslinking and Immuno-precipitation (PAR-CLIP);
(iv) RNA immunopurification followed by microarray hybridization (RIP-chip);
(v) RNA immunopurification followed by high throughput sequencing (RIP-seq);
(vi) RNA-protein crosslink;
(vii) RNA pulldown;
(viii) Mass-spectrometry;
(ix) Proximity Extension Assay;
(x) Immunofluorescent based assays;
(xi) Proximity Ligation Assay;
(xii) Förster resonance energy transfer (FRET), and/or
(xiii) any other method for reporting at least one bi-molecular interaction.

6. The method according to any one of claims 1 to 5, wherein the at least one enzyme of the glycolytic pathway is Enolase 1 (ENO1), or a derivative, a precursor, a mutant, or a functional fragment thereof, comprising the amino acid sequence according to SEQ ID NO: 1, or an amino acid sequence having at least 80%, preferably at least 90%, more preferably at least 95%, and most preferably at least 99% sequence identity to SEQ ID NO: 1.

7. A mutated Enolase 1 (ENO1) enzyme, or a functional fragment thereof, wherein the mutated ENO1 enzyme amino acid sequence when aligned to the amino acid sequence of SEQ ID NO: 1 comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to SEQ ID NO: 1.

8. The mutated ENO1 enzyme, or the functional fragment thereof, according to claim 7, wherein the amino acid sequence of the mutated ENO1 enzyme, or of the functional fragment thereof, when aligned to the amino acid sequence of SEQ ID NO: 1, comprises at least one amino acid substitution, deletion, and/or addition in an amino acid at positions 57-132 of SEQ ID NO: 1, or at position 343 of SEQ ID NO: 1, preferably wherein the amino acid sequence of the mutated ENO1 enzyme, or of the functional fragment thereof, comprises at least one amino acid substitution, deletion, and/or addition at position 89, 92, 105, and/or 343 of SEQ ID NO: 1, and more preferably comprises the amino acid sequence of any of SEQ ID NOs: 7 to 14, or comprises not more than 50 amino acid substitutions, deletions, and/or additions, preferably not more than 40, more preferably not more than 3o, even more preferably not more than 10, even more preferably not more than 5, even more preferably not more than 4, even more preferably not more than 2, and most preferably not more than 1 amino acid substitution, deletion, and/or addition of an amino acid sequence according to the sequence of any of SEQ ID NOs: 7 to 14.

9. The mutated ENO1 enzyme, or the functional fragment thereof, according to claim 7 or 8, comprising at least one amino acid substitution selected from K89A, K92A, and Kio5A at positions 89, 92, and 105 in SEQ ID NO: 1, or comprising the amino acid substitutions K89A and K92A at positions 89, and 92 in SEQ ID NO: 1, or comprising the amino acid substitutions K89A and K105A at positions 89, and 105 in SEQ ID NO: 1, or comprising the amino acid substitutions K92A and K105A at positions 92, and 105 in SEQ ID NO: 1, or comprising the amino acid substitutions K89A, K92A, and K105A at positions 89, 92, and 105 in SEQ ID NO: 1, wherein said mutated ENO1 enzyme is characterized by an enhanced binding of at least one nucleic acid to the mutated ENO1 enzyme compared to the binding of the at least one nucleic acid to the wild type ENO1 enzyme comprising the amino acid sequence of SEQ ID NO: 1, preferably wherein the mutated ENO1 enzyme comprises the amino acid sequence of any of SEQ ID NOs: 7 to 13, or comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to the sequence of any of SEQ ID NOs: 7 to 13.

10. The mutated ENO1 enzyme, or the functional fragment thereof, according to any one of claims 7 to 9, comprising at least one amino acid substitution at position 343 in SEQ ID NO: 1, such as a K343A amino acid substitution at position 343 in SEQ ID NO: 1, wherein said mutated ENO1 enzyme is characterized by a reduced binding of at least one nucleic acid to the mutated ENO1 enzyme compared to the binding of the at least one nucleic acid to the wild type ENO1 enzyme comprising the amino acid sequence of SEQ ID NO: 1, preferably wherein the mutated ENO1 enzyme comprises the amino acid sequence of SEQ ID NO: 14, or comprises not more than 50 amino acid substitutions, deletions, and/or additions, preferably not more than 40, more preferably not more than 30, even more preferably not more than 10, even more preferably not more than 5, even more preferably not more than 4, even more preferably not more than 2, and most preferably not more than 1 amino acid substitution, deletion, and/or addition of an amino acid sequence according to SEQ ID NO: 14.

11. An isolated nucleic acid, comprising a sequence coding for the mutated ENO1 enzyme, or the functional fragment thereof, according to any one of claims 7 to 10, or a vector, comprising the nucleic acid, optionally wherein the vector is an expression vector, comprising a promoter sequence operably linked to the nucleic acid.

12. A recombinant cell comprising a mutated ENO1 enzyme, or the functional fragment thereof, according to any one of claims 7 to 10, or a nucleic acid or a vector according to claim 11.

13. A pharmaceutical composition comprising the mutated ENO1 enzyme, or the functional fragment thereof, according to any one of claims 7 to 10, a nucleic acid or a vector according to claim 11, or a recombinant cell according to claim 12, together with a pharmaceutically acceptable carrier, stabilizer and/or excipient.

14. A compound for use in the treatment of a disease, the compound being selected from a mutated ENO1 enzyme, or the functional fragment thereof, according to any one of claims 7 to 10, a nucleic acid or a vector according to claim 11, a recombinant cell according to claim 12, and a pharmaceutical composition according to claim 13, wherein the disease is preferably a proliferative disease, such as cancer, diabetes, an infectious disease, a metabolic disease, an immune-related disease, a degenerative disease, such as a neurodegenerative disease, for example Alzheimer's disease, and/or aging.

15. A method for identifying and/or characterizing a compound suitable for the prevention and/or treatment of a disease, the method comprising the steps of:
(a) Providing at least one enzyme of the glycolytic pathway, and a candidate compound;
(b) Bringing into contact the at least one enzyme of the glycolytic pathway, and the candidate compound;
(c) Detecting and/or quantifying at least one modification in the at least one enzyme of the glycolytic pathway;
wherein a differential level of the at least one modification in the at least one enzyme of the glycolytic pathway contacted with the candidate compound compared to the at least one modification in the at least one enzyme of the glycolytic pathway not contacted with the candidate compound indicates the candidate compound as suitable for the prevention and/or treatment of the disease, and wherein the differential level of the at least one modification is indicative for a differential level of a binding of the at least one enzyme of the glycolytic pathway to at least one nucleic acid, preferably wherein the modification is selected from ubiquitination, acetylation, phosphorylation, methylation, glycosylation, lipid-conjugation, functionalization, heterodimerization, homodimerization, oxidation, hydroxylation, or any other natural or artificial post-translational modification, or combinations thereof.

16. A method for diagnosing, prognosing, stratifying and/or monitoring of a therapy, of a disease in a subject, comprising the steps of:
(a) Providing a sample comprising at least one enzyme of the glycolytic pathway from the subject, at least one nucleic acid, and optionally at least one agent for detection of the at least one enzyme of the glycolytic pathway, the at least one nucleic acid, and/or a binding between the at least one enzyme of the glycolytic pathway and the at least one nucleic acid, such as an antigen binding construct (for example, an antibody, an antibody-like molecule or other antigen binding derivative, or an antigen binding fragment thereof), a nucleic acid, including an RNA or DNA aptamer, and the like;
(b) Optionally, isolating the at least one enzyme of the glycolytic pathway from the sample;
(c) Bringing into contact the at least one enzyme of the glycolytic pathway, and the at least one nucleic acid, and
(d) Detecting and/or quantifying the binding between the at least one enzyme of the glycolytic pathway and the at least one nucleic acid;
wherein a differential level of the binding between the at least one enzyme of the glycolytic pathway and the at least one nucleic acid in the sample from the subject as detected and/or quantified in step (d) compared to a control or reference value is indicative for the diagnosis, prognosis, stratification and/or monitoring of a therapy, of the disease in the subject.


SEQUENCE LISTING (14 sequences)

SEQ ID NO: 1 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 2 - DNA, 13 bp, Homo sapiens
ttttttbtttttt

SEQ ID NO: 3 - DNA, 7 bp, Homo sapiens
cccagrc

SEQ ID NO: 4 - protein, 636 aa, Homo sapiens
MNPSAPSYPMASLYVGDLHPDVTEAMLYEKFSPAGPILSIRVCRDMITRRSLGYAYVNFQQPADAERALDTMNFDVIKGKPVRIMWSQRDPSLRKSGVGNIFIKNLDKSIDNKALYDTFSAFGNILSCKVVCDENGSKGYGFVHFETQEAAERAIEKMNGMLLNDRKVFVGRFKSRKEREAELGARAKEFTNVYIKNFGEDMDDERLKDLFGKFGPALSVKVMTDESGKSKGFGFVSFERHEDAQKAVDEMNGKELNGKQIYVGRAQKKVERQTELKRKFEQMKQDRITRYQGVNLYVKNLDDGIDDERLRKEFSPFGTITSAKVMMEGGRSKGFGFVCFSSPEEATKAVTEMNGRIVATKPLYVALAQRKEERQAHLTNQYMQRMASVRAVPNPVINPYQPAPPSGYFMAAIPQTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 5 - protein, 183 aa, Homo sapiens
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 6 - protein, 173 aa, Homo sapiens
MARMNRPAPVEVTYKNMRFLITHNPTNATLNKFIEELKKYGVTTIVRVCEATYDTTLVEKEGIHVLDWPFDDGAPPSNQIVDDWLSLVKIKFREEPGCCIAVHCVAGLGRAPVLVALALIEGGMKYEDAVQFIRQKRRGAFNSKQLLYLEKYRPKMRLRFKDSNGHRNNCCIQ

SEQ ID NO: 7 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEAIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 8 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDALMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 9 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSAFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 10 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEAIDALMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 11 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEAIDKLMIEMDGTENKSAFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 12 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDALMIEMDGTENKSAFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 13 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEAIDALMIEMDGTENKSAFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 14 - protein, 434 aa, Homo sapiens
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLAVNQIGSVTESLQACKLAQANGWGVMVSHRSGETEDTFIADLVVGLCTGQIKTGAPCRSERLAKYNQLLRIEEELGSKAKFAGRNFRNPLAK